(19) United States
(12) Patent Application Publication
(10) Pub. No.: US 2026/0234717 A1
(43) Pub. Date: Aug. 13, 2026

(54) DETECTION OF MODIFIED NUCLEOBASES IN NUCLEIC ACID SAMPLES

(71) Applicant: Roche Sequencing Solutions, Inc., Pleasanton, CA (US)

(72) Inventors: Robert Busam, Seattle, WA (US); Marc Prindle, Seattle, WA (US); John Tabone, Kirkland, WA (US); Alexander Lehmann, Seattle, WA (US); Jagadeeswaran Chandrasekar, Seattle, WA (US); Mark Stamatios Kokoris, Bothell, WA (US); Robert N. McRuer, Bainbridge Island, WA (US); Joseph Horsman, Seattle, WA (US); Svetlana Kritzer, Seattle, WA (US); Grant Kingsley, Seattle, WA (US); Aaron Jacobs, Seattle, WA (US)

(21) Appl. No.: 19/180,252

(22) Filed: Apr. 16, 2025

Related U.S. Application Data
(63) Continuation of application No. PCT/EP2023/079149, filed on Oct. 19, 2023.
(60) Provisional application No. 63/380,439, filed on Oct. 21, 2022.

(51) Int. Cl. C12Q 1/6874; C12Q 1/6806

(52) U.S. Cl. CPC C12Q 1/6874; C12Q 1/6806

(57) ABSTRACT

Described are methods of detecting modified nucleotide bases in a nucleic acid sample using specific DNA glycosylases to excise a modified nucleobase of interest. Prior to glycosylase treatment, DNA target fragment templates are copied by a DNA polymerase to produce a first complementary copy strand that preserves the genetic information of the DNA target fragment. Following glycosylase treatment, the DNA target fragment templates are copied by an abasic bypass polymerase to produce a second complementary copy strand that preserves the epigenetic information of the DNA target fragment. Comparison of the DNA sequences of the two complementary copy strands enables identification of the positions of the modified nucleobases in the DNA target fragment.

### CROSS REFERENCE TO RELATED APPLICATIONS

This patent application is a continuation of International Patent Application No. PCT/EP2023/079149, filed Oct. 19, 2023, which claims priority to and the benefit of U.S. Provisional Application No. 63/380,439, filed Oct. 21, 2022. Each of the above patent applications is incorporated herein by reference as if set forth in its entirety.

### SEQUENCE LISTING INCORPORATION BY REFERENCE

This application contains a sequence listing, which has been filed electronically in xml format and is herein incorporated by reference in its entirety. Said xml copy has a file name of P37864-US-1_Sequence_Listing.xml, was created on Apr. 15, 2025, and is 5120 bytes in size.

### BACKGROUND

Methylation and the products of various forms of DNA damage have been implicated in a variety of important biological processes. Changes in methylation patterns and the appearance of damaged DNA are often among the earliest events observed for various disease states

Epigenetic modifications are essential for normal development. For example, methylcytosine, the most widely studied epigenetic modification, is associated with a number of key processes including genomic imprinting, X-chromosome inactivation, suppression of repetitive elements, and carcinogenesis. For example, DNA methylation at the 5 position of cytosine has the specific effect of reducing gene expression and has been found in every vertebrate examined. In many disease processes, such as cancer, gene promoter CpG islands acquire abnormal hypermethylation, which results in transcriptional silencing that can be inherited by daughter cells following cell division. In addition, alterations of DNA methylation have been recognized as an important component of cancer development. Hypomethylation, in general, arises earlier and is linked to chromosomal instability and loss of imprinting, whereas hypermethylation is associated with promoters and can arise secondary to gene (oncogene suppressor) silencing. Additionally, hydroxymethylcytosine has also emerged as an important epigenetic modification as well with potential regulatory roles in gene expression ranging from development to aging. Various cancers have shown that hydroxymethylcytosine content is consistently and significantly reduced in malignant versus healthy tissues, even in early-stage lesions.

DNA is under constant stress from both endogenous and exogenous sources. The bases exhibit limited chemical stability and are vulnerable to chemical modifications through different types of damage, including oxidation, alkylation, radiation damage, and hydrolysis. Damage to DNA bases may affect their base-pairing properties and, therefore, may be mutagenic. DNA base modifications resulting from these types of DNA damage are wide-spread and play important roles in affecting physiological states and disease phenotypes. Examples include 7,8-dihydro-8-oxoguanine (8-oxoG) (oxidative damage), 8-oxoadenine (oxidative damage; aging, Alzheimer's, Parkinson's), 1-methyladenine, 06-methylguanine (alkylation; gliomas and colorectal carcinomas), benzo[a]pyrene diol epoxide (BPDE), pyrimidine dimers (adduct formation; smoking, industrial chemical exposure, UV light exposure; lung and skin cancer), and 5-hydroxy cytosine, 5-hydroxyuracil, 5-hydroxymethyluracil, and thymine glycol (ionizing radiation damage; chronic inflammatory diseases, prostate, breast and colorectal cancer). For example, 8-oxoG is a frequent product of DNA oxidation. 8-oxoG tends to base-pair with adenine, giving rise to GC to T A transversion mutations. Another example is the hydrolytic deamination of cytosine and 5-methylcytosine (5-meC) to give rise to uracil and thymine mis-paired with guanine, respectively, causing CG to T A transition mutations if not repaired. In another example, alkylation can generate a variety of DNA base lesions comprising 6-meG, N7-methylguanine (7-meG), or N3-methyladenine (3-meA). While 6-meG is promutagenic by its property to pair with thymine, 7-meG and 3-meA block replicative DNA polymerases and are therefore cytotoxic. These and many other forms of DNA base damage arise in cells many times every day and only the continuous action of specialized DNA repair systems can prevent a rapid decay of genetic information. In addition to damage to nuclear DNA, mitochondrial DNA also experience significant oxidative damage, as well as damage from alkylation, hydrolysis, and adducts. For example, oxidative damage is the most prevalent type of damage in mitochondrial DNA, primarily because mitochondria are a major cellular source of reactive oxygen species (ROS). In addition, mitochondria house approximately 30% of the cellular pool of S-adenosylmethionine, which can methylate DNA nonenzymatically. Also, exposure to certain agents, such as estrogens, tobacco smoke, and certain chemicals, leads to preferential damage of mitochondrial DNA.

As DNA damage and epigenetic modification may be the earliest indications of disease state, detection of epigenetic modification and DNA damage patterns can be useful for early detection of disease and intervention. However, detection methods have limitations. For example, with respect to methylation status, spectrophotometry can be used to indicate global content of a modification in target DNA, but has limited specificity. High-performance liquid chromatography (HPLC) and mass spectrometry are also often used, but are costly, require significant amounts of material, and reduce DNA to constituent nucleosides or nucleotides, thus destroying sequence information for downstream analysis. Immunoprecipitation (IP) using monoclonal antibodies can enrich DNA with target modifications, but limitations with specificity have been identified. Restriction digest profiling utilizes fragment analysis of DNA treated with modification-sensitive restriction endonucleases, but requires large amounts of material and is limited to sequences featuring a restriction site with known sensitivity. While bisulfite sequencing is considered the “gold-standard” technique for detection of DNA methylation, there are important limitations. First, the chemical conversion process causes widespread non-specific damage to DNA, and thus the approach requires large amounts of starting material. Second, the method can be expensive and time consuming, requiring multiple sequencing runs. Finally, and importantly, it is generally only applicable to methylcytosine (mC) modifications. Variations have been developed or suggested that allow a limited number of additional modification types to be targeted (methylcytosine (mC) and hydroxymethylcytosine (hmC)) but these are low-yield and still share the other limitations listed above. They are also not readily applicable to other modifications and are fairly complex.

Thus, there is a need in the art for improved methods of detecting modified nucleobases in DNA samples of interest. The present invention fulfills these needs and provides further related advantages as discussed below.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### BRIEF SUMMARY OF THE INVENTION

Aspects of the present invention encompass detection of modified nucleobases, such as epigenetic changes and DNA damage, in DNA samples.

In one aspect, the invention provides a method of identifying a modified nucleobase in a plurality of nucleic acids, the method including the steps of: providing a sample including a plurality of DNA templates; generating first complementary copies of the DNA templates, the generating being directed by an oligonucleotide primer using a first DNA polymerase in the presence of native dNTPs, in which the generating produces a complementary copy of each of the DNA templates such that each complementary copy comprises native dNTPs, and in which each complementary copy is hybridized to one of the DNA templates; subjecting the DNA templates and the first complementary copies to DNA glycosylase treatment, in which the DNA glycosylase specifically excises the modified nucleobase in the DNA templates to convert the positions of the modified nucleobases into abasic sites, resulting in each glycosylase-converted DNA template being hybridized to a non-converted complementary copy; generating a second complementary copy of the glycosylase-converted DNA templates, the generating being directed by a second DNA polymerase, in which the second DNA polymerase is capable of incorporating a nucleotide opposite the abasic sites in the converted DNA templates, wherein the nucleotide does not Watson-Crick base pair with the modified nucleobase; determining the nucleotide sequence of the first and second complementary copies; and comparing the nucleotide sequence of the second complementary copies to the nucleotide sequence of the first complementary copies for each of the DNA glycosylase-converted DNA templates, thereby determining the positions of the modified nucleobase in the DNA templates prior to DNA glycosylase conversion.

In one embodiment, the step of comparing the nucleotide sequence of the second complementary copies to the nucleotide sequence of the first complementary copies for each of the DNA glycosylase-converted DNA templates identifies a nucleotide substitution in the sequence of the second complementary copies relative to the first complementary copies, in which the position of the nucleotide substitution identifies the position of the modified base in the DNA templates. In another embodiment, the modified nucleobase is selected from 5-mC, 5-hmC, 5-fC, and 5-caC. In another embodiment, the DNA glycosylase is a monofunctional DNA glycosylase. In yet another embodiment, the monofunctional DNA glycosylase is thymine DNA glycosylase (TDG), or a variant thereof. In another embodiment, the step of subjecting the DNA templates and the first complementary copies to DNA glycosylase treatment further includes subjecting the DNA templates and the first complementary copies to treatment with a ten eleven translocation (TET) enzyme, or a variant thereof. In yet another embodiment, the ten eleven translocation (TET) enzyme, or a variant thereof, is ngTET. In another embodiment, the DNA glycosylase is a bifunctional DNA glycosylase. In yet another embodiment, the bifunctional DNA glycosylase is member of the DEMETER (DME) family of DNA glycosylases, or a variant thereof. In yet another embodiment, the member of the DEMETER (DME) family of DNA glycosylases, or a variant thereof, is a variant engineered to inactivate lyase activity. In another embodiment, the second DNA polymerase is an abasic bypass DNA polymerase. In yet another embodiment, the abasic bypass DNA polymerase is a DPO4 polymerase, or variant thereof. In yet another embodiment, the DPO4 polymerase, or variant thereof, is a variant including the following mutations: M76W, K78E, E79P, Q82W, Q83G, and S86E (SEQ ID NO:3). In another embodiment, the abasic bypass DNA polymerase incorporates dATP in the second complementary copies at positions opposite the abasic sites in the glycosylase-converted DNA templates. In another embodiment, the abasic bypass DNA polymerase further includes a third DNA polymerase, in which the third DNA polymerase has exonuclease activity. In yet another embodiment, the third DNA polymerase is a DPO1 polymerase. In another embodiment, the first DNA polymerase is a high-fidelity DNA polymerase. In another embodiment, the method further includes the step of treating the glycosylase-converted DNA templates with a stabilizing agent prior to the step of generating the second complementary copies of the glycosylase-converted DNA templates. In another embodiment, the stabilizing agent includes an aldehyde-reactive compound that forms a stable adduct with the abasic sites. In yet another embodiment, the stabilizing agent is selected from O-hydroxylamines, acyl hydrazines, tryptamines, beta amino thiols, alkyl hydrazines, hydrazino-iso-pictet-spengler indoles, and methylaminooxy-iso-pictet-spengler indoles. In another embodiment, the stabilizing agent includes an aminoxyalkyl group capable of forming an oxime adduct with the abasic site. In yet another embodiment, the stabilizing agent is selected from 1-[2-(amino)ethyl]-uracil, 1-[3-(aminoxy)propyl]-uracil, 1-[4-(aminoxy)butyl]-uracil, 1-[5-(aminoxy)pentyl]-uracil, 1-[2-(aminoxy)ethyl]-2,4-diiodo-5-methyl benzene, 1-[2-(aminoxy)ethyl]-2,4-dibromo-5-methyl benzene, 1-[2-(aminoxy)ethyl]-2,4-dichloro-5-methyl benzene, 1-[2-(aminoxy)ethyl]-2,4-difluoro-5-methyl benzene, and 1-[2-(aminoxy)ethyl]-thymine. In yet another embodiment, the stabilizing agent is 1-[2-(amino)ethyl]-uracil. In another embodiment, the step of subjecting the DNA templates and the first complementary copies to DNA glycosylase treatment and the step of the treating the glycosylase-converted DNA templates with a stabilizing agent prior to generating the second complementary copies occur in the same step. In another embodiment, the DNA templates are selected from the group consisting of genomic DNA, mitochondrial DNA, cell-free DNA, circulating tumor DNA, or combinations thereof. In another embodiment, the DNA templates are immobilized on a solid support. In yet another embodiment, the first or second complementary copies are immobilized on a solid support. In another embodiment, the step of determining the nucleotide sequences of the first and second complementary copies includes the steps of synthesizing an Xpandomer copy of the first and second complementary copies and passing the Xpandomer copies of the first and second complementary copies through a nanopore. In another embodiment, the DNA templates include a first adapter joined to the 5′ end of the DNA template and a second adapter joined to the 3′ end of the template. In yet another embodiment, the first or second adapters are Y adapters. In yet another embodiment, at least one of the first and second adapters includes a unique molecular identifier barcode (UMI). In another embodiment, the step of comparing the sequences of the first and second complementary copies includes bioinformatically pairing sequences including the same unique molecular identifier barcode (UMI).

In another aspect, the invention provides a chemo-enzymatic nucleobase conversion reaction mixture including a DNA glycosylase enzyme, a chemical stabilizing agent, and a suitable buffer. In one embodiment, the chemo-enzymatic nucleobase conversion reaction mixture further includes a DNA template strand hybridized to a first complementary copy strand, in which the DNA template strand includes a modified nucleobase and the first complementary copy strand comprises native nucleobases. In another embodiment, the chemical stabilizing agent includes an aminoxyalkyl group, in which the aminoxyalkyl group is capable of reacting with an abasic nucleotide including an open-ring aldehyde moiety to form a stable oxime adduct. In another embodiment, the chemical stabilizing agent is selected from 1-[2-(amino)ethyl]-uracil, 1-[3-(aminoxy)propyl]-uracil, 1-[4-(aminoxy)butyl]-uracil, 1-[5-(aminoxy)pentyl]-uracil, 1-[2-(aminoxy)ethyl]-2,4-diiodo-5-methyl benzene, 1-[2-(aminoxy)ethyl]-2,4-dibromo-5-methyl benzene, 1-[2-(aminoxy)ethyl]-2,4-dichloro-5-methyl benzene, 1-[2-(aminoxy)ethyl]-2,4-difluoro-5-methyl benzene, and 1-[2-(aminoxy)ethyl]-thymine. In another embodiment, the chemical stabilizing agent is selected from O-hydroxylamines, acyl hydrazines, tryptamines, beta amino thiols, alkyl hydrazines, hydrazino-iso-pictet-spengler indoles, and methylaminooxy-iso-pictet-spengler indoles. In another embodiment, the DNA glycosylase is selected from the DNA glycosylases set forth in Table 1. In yet another embodiment, the reaction mixture includes more than one DNA glycosylase set forth in Table 1. In yet another embodiment, DNA glycosylase is TDG, or a variant thereof. In another embodiment, the reaction mixture further includes a TET enzyme.

In another aspect, the invention provides a kit for the detection of a modified nucleobase in a DNA sample, including any of the above chemo-enzymatic nucleobase conversion reaction mixtures, an enzyme selected from at least one of a high-fidelity DNA polymerase, an abasic bypass DNA polymerase, and a DNA polymerase with exonuclease activity and a suitable mixture of dNTPs, or analogs thereof. In one embodiment, the kit further includes one or more of a buffer for the enzyme.

### BRIEF DESCRIPTION OF THE DRAWINGS

FIG. 1A, FIG. 1B, FIG. 1C and FIG. 1D are condensed schematics summarizing one embodiment of the methods of the present invention.

FIG. 2A and FIG. 2B are schematics illustrating alternative embodiments of solid-state synthesis of primer extension products.

FIG. 3A and FIG. 3B are chemical schemes illustrating the instability of abasic sites and one embodiment of a means to stabilize abasic sites.

FIG. 4A and FIG. 4B are schemes illustrating two exemplary enzymatic reactions for excising 5-mC nucleobases to produce abasic sites in a DNA target fragment.

FIG. 5A and FIG. 5B provide exemplary embodiments of chemical schemes for the stabilization of abasic sites in a converted DNA target fragment.

FIG. 6A and FIG. 6B are schemes illustrating one embodiment of aminoxyalkyl-mediated “hijack” of DNA lyase activity.

FIG. 7 provides the chemical structures of certain exemplary nucleotide analogs for the practice of the methods of the invention.

FIG. 8 provides the chemical structures of other exemplary nucleotide analogs for the practice of the methods of the invention.

FIG. 9 provides the chemical structures of other exemplary nucleotide analogs for the practice of the methods of the invention.

FIG. 10 provides the chemical structures of other exemplary nucleotide analogs for the practice of the methods of the invention.

FIG. 11 provides the chemical structures of certain exemplary generic nucleotide analogs for the practice of the methods of the invention.

FIG. 12A and FIG. 12B are schemes illustrating one embodiment of chemo-enzymatic conversion of a DNA target fragment including a modified nucleobase of interest (5-mC) using an exemplary aminoxyalkyl uracil mimetic to stabilize the abasic site in the DNA target fragment and use thereof for identification of the modified nucleobase in the DNA target fragment by Sequencing by Expansion.

FIG. 13 provides the chemical structures of certain exemplary aminoxyalkyl nucleobase mimetics for the practice of the methods of the invention.

FIG. 14 is a gel showing the DNA products of certain chemo-enzymatic nucleobase conversion reactions.

FIG. 15 is a gel showing the DNA products of certain primer extension reactions of an abasic DNA template with DPO4 polymerase.

FIG. 16 is a gel showing the DNA products of certain primer extension reactions of an abasic template using various DNA polymerases and combinations thereof.

FIG. 17A and FIG. 17B are graphs showing the nucleotides incorporated by a DNA polymerase at positions opposite abasic sites in a converted DNA template stabilized by a uracil nucleobase mimetic and nucleotides incorporated opposite 5-mC in an unconverted template, respectively, as determined by DNA sequencing of the templates.

### DETAILED DESCRIPTION OF THE INVENTION

The present invention may be understood more readily by reference to the following detailed description of preferred embodiments of the invention and the Examples included herein. Unless otherwise explained, all technical and scientific terms used herein have the same meaning as commonly understood by one of ordinary skill in the art to which this disclosure belongs.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include”, as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

The abbreviation, “e.g.,” is derived from the Latin exempli gratia, and is used herein to indicate a non-limiting example. Thus, the abbreviation “e.g.,” is synonymous with the term “for example.” It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicants reserve the right to revise the application or claims to refer specifically to any individual member or any subgroup of members of the Markush group.

Any headings used within this document are only being utilized to expedite its review by the reader, and should not be construed as limiting the invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated.

### Methods of Detecting Modified DNA Nucleobases

Described herein are methods and compositions for the detection of modified nucleobases in DNA samples, reflecting, for example, epigenetic modifications and DNA damage. The methods include enzymatic excision of a modified nucleobase of interest in a DNA target fragment to produce an abasic site at each position in which the modified nucleobase of interest occurs in the nucleic acid sequence of the DNA target fragment. The positions of the abasic sites may be identified by DNA sequencing methodologies, as described herein. The methods of the present invention also include a workflow that generates a first complementary copy and a second complementary copy of a DNA target fragment template (i.e., a first daughter strand and a second daughter strand). The first complementary copy is generated before enzymatic excision of the modified nucleobase of interest, while the second complementary copy is generated after enzymatic excision of the modified nucleobase of interest. The first and second complementary copies thus encode the genetic and, e.g., epigenetic information of the DNA target fragment, respectively. Sequence information obtained from the first and second complementary copies can be compared to identify the positions of the modified nucleobase of interest in the nucleic acid sequence of the original DNA target fragment.

### Overview

According to the methods described herein, a modified nucleobase of interest may include, but not necessarily be limited to, one or more of 5-methylcytosine (5-mC), 5-hydroxymethylcytosine (5-hmC), 5-carboxycytosine (5-caC), 5-formylcytosine (5-fC), 8-oxo-7,8-dihyroguanine (*-oxoG), uracil (U), 6-methyladenine (6-mA), 8-oxoadenine, O-6-methylguanine, 1-methyladenine, O-4-methylthymine, 5-hydroxycytosine, 5-hydroxyuracil, 5-hydroxymethyluracil, or thymine dimers. In some instances, a plurality of any combination of these exemplary, and other, modified nucleobases may be detected by the methods of the present invention.

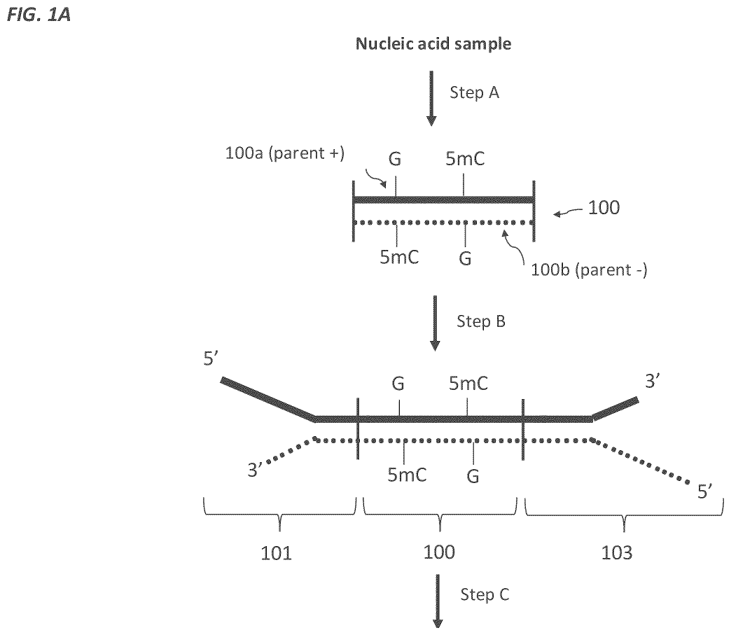

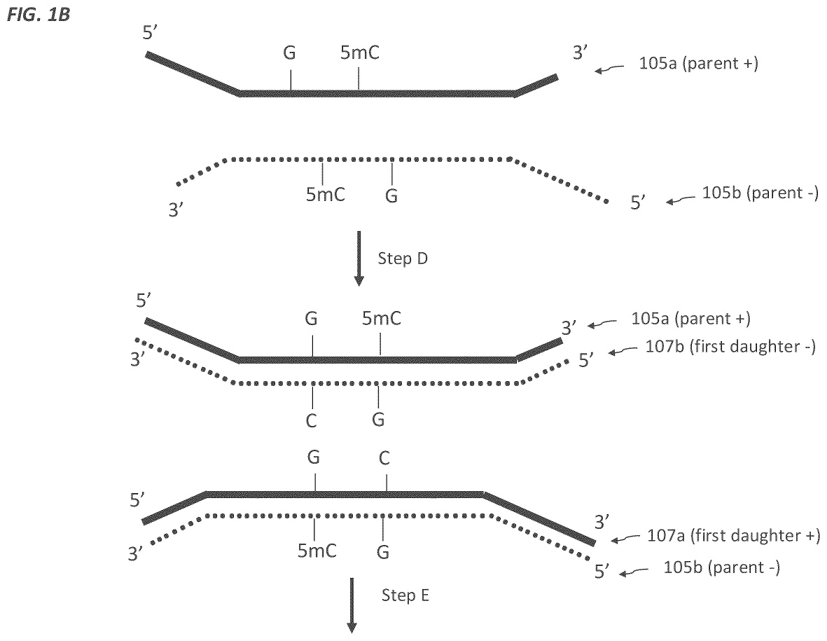

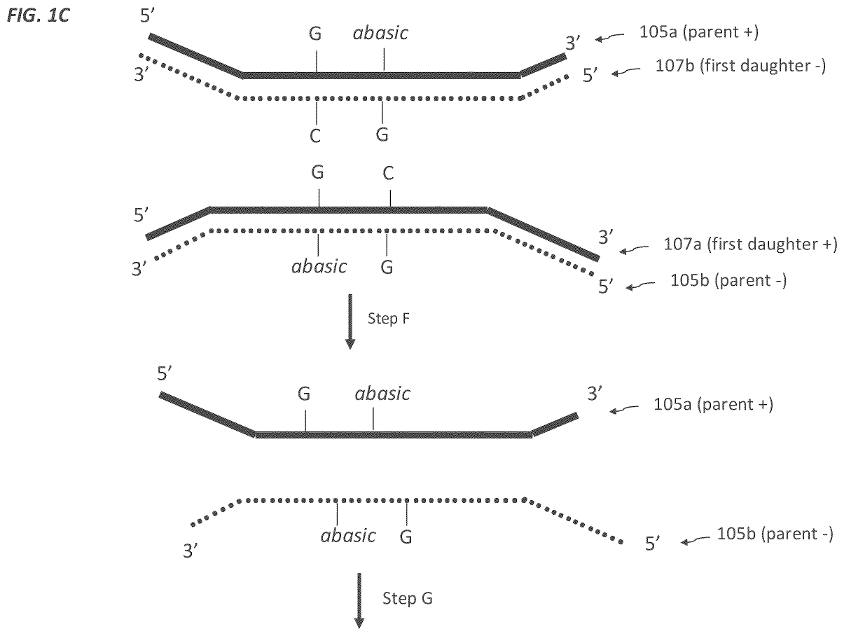

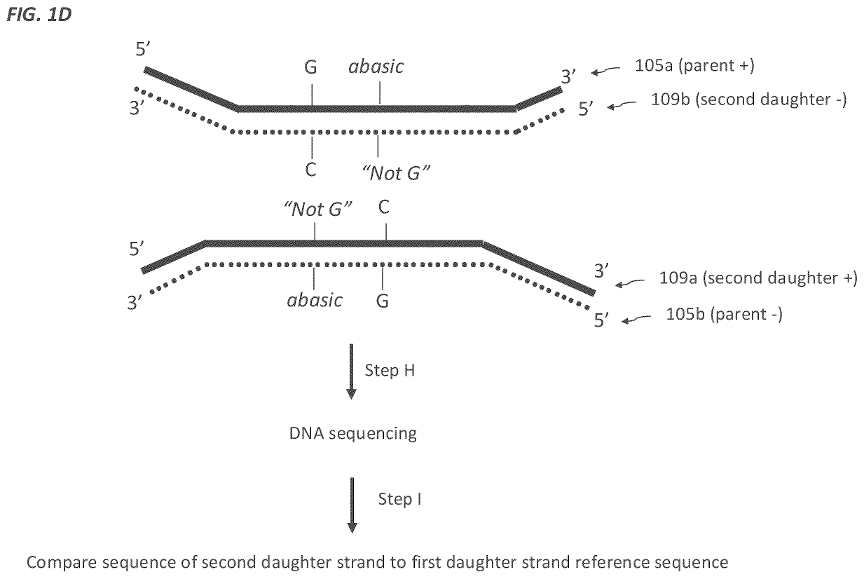

In one aspect, methods are provided for detecting a modified nucleobase, e.g., a modified nucleobase of interest, in a sample of nucleic acids. A schematic overview of one exemplary method is illustrated in FIGS. 1A-1D. The method may include Step A of obtaining a sample of nucleic acids and fragmenting the nucleic acids to produce a sample that includes DNA target fragments 100. As used herein, the term “target fragment” means that the corresponding nucleic acid fragment is derived from a biological sample and is a target for the methods described herein, which interrogate nucleic acid sequences for the presence of a particular modified nucleobase. In this non-limiting depiction, a modified nucleobase of interest is methylated cytosine (5-mC) and the DNA target fragment is a double stranded nucleic acid fragment. Here, the stands of the DNA target fragment are depicted as “parent (+)” 100a (i.e., the sense strand) and “parent (−)” 100b (i.e., the antisense strand). For simplicity, each of the strands of the DNA target fragment in this example includes a single 5-mC residue.

In some instances, the DNA target fragment may be genomic DNA, mitochondrial DNA, cell free DNA (cfDNA), circulating tumor DNA (ctDNA), or a combination thereof, obtained from a biological sample.

In certain embodiments, the method may then include Step B of ligating (i.e., joining) adapters 101 and 103 to the 5′ and 3′ ends of the DNA target fragments to produce adapter-ligated DNA target fragments. The adapters may include a region of double stranded DNA and a region of single stranded DNA. In the example depicted in FIG. 1A, the adaptors are Y adapters (YAD) and include a double stranded region and two regions of single stranded DNA. The adapters may also include sequences, or other features, that mediate downstream steps of the workflow. For example, in certain embodiments, the adapters may include sequences for immobilization of the adaptor-ligated DNA target fragments on a solid support, sequences for hybridization of oligonucleotide primer(s), sequences enabling bioinformatic analysis of DNA sequence information (e.g., unique molecular identifier bar codes [UMI], sample identifiers [SID]), chemical moieties for solid-phase immobilization and the like. In certain embodiments, the structures of adapters 101 and 103 may be identical or different, depending on the particular application.

The method may then include Step C of denaturing the DNA target fragments to produce single stranded parent (+) strand 105a and single stranded parent (−) strand 105b. As used herein, the terms “target” and “parent” are used interchangeably as they relate to strands of nucleic acids. Further, as used herein, the single stranded DNA target fragments may be referred to interchangeably as “DNA templates”, which refers to a strand of a polynucleotide from which a complementary polynucleotide can be hybridized or synthesized by a nucleic acid polymerase, for example, in a primer extension reaction.

The method may then include Step D of performing a first primer extension reaction. The first primer extension reaction is directed by an extension oligonucleotide (i.e., a primer), hybridized to the DNA template using a first DNA polymerase. In some instances, the extension oligonucleotide may hybridize to a region in an adapter sequence. The first primer extension reaction produces a sample of double stranded DNA fragments, each including a newly synthesized first complementary copy strand (i.e., first daughter strands 107b and 107b) hybridized (i.e., coupled) to the target fragment template (i.e., parent strands 105a and 105b). In some instances, the first DNA polymerase is a high-fidelity DNA polymerase. In this step, the sample of double stranded DNA fragments is distinguished from the sample of DNA target fragments of Step A in that it includes a complementary copy strand that is synthesized in vitro. The primer extension reaction may be carried out under conditions in which the complementary copy strands produced are “native” strands in that they do not include the modified nucleobase(s) of interest present in the target strands. For example, in this depiction, the first complementary copy strands incorporate native cytosine residues at the positions of methylated cytosine residues in the corresponding target strands. As used herein, the term “native” refers to a nucleobase, nucleotide, or polynucleotide that is analogous to a related modified nucleobase, nucleotide, or polynucleotide except for the specific modification of the modified nucleobase, nucleotide, or polynucleotide. Thus, in certain aspects, each modified nucleobase, nucleotide, or polynucleotide can have an analogous native nucleobase, nucleotide, or polynucleotide, and vice versa.

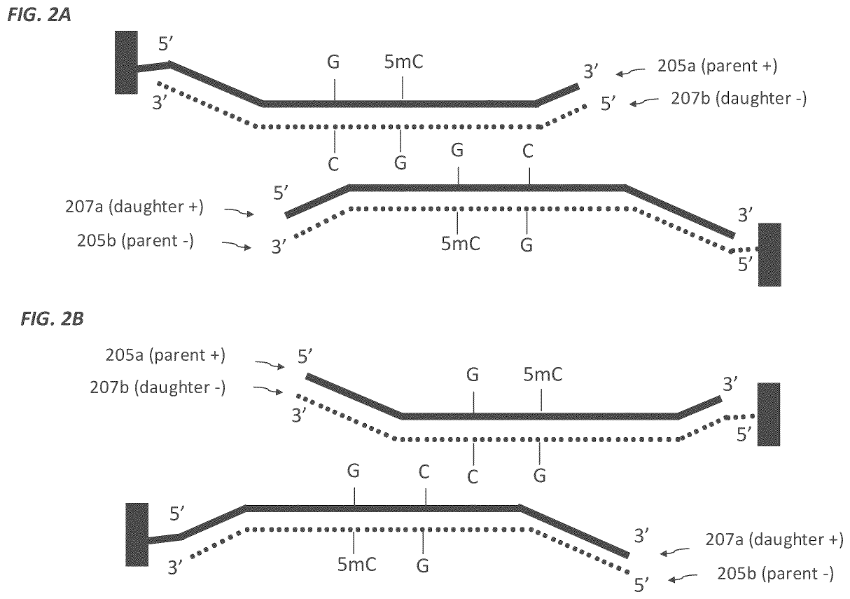

In some instances, the target fragment templates are immobilized on a solid support prior to the step (D) of performing the first primer extension reaction, as depicted in FIG. 2A. As illustrated here, the newly synthesized complementary copy strands are not immobilized on the solid support and may be physically separated from the immobilized template strands upon denaturation of the double stranded DNA fragments. In other instances, as depicted in FIG. 2B, an oligonucleotide complementary to the template strand, e.g., to the adapter sequence, is immobilized on a solid support and is capable of “capturing” the template strand via hybridization. Following capture of the target fragment, the first primer extension reaction may be performed, using the hybridized oligonucleotide as a primer, to produce the first complementary copy strand likewise immobilized on the solid support. In this instance, denaturation of the resulting double stranded DNA fragment will release the template strand from the solid support, while retaining the complementary copy.

The methods may then include Step E of treating the sample of double stranded DNA fragments with a DNA glycosylase enzyme capable of excising the modified nucleobase of interest (e.g., 5-mC in this depiction). As used herein, the term “excise” means cleaving the N-glycosidic bond between the sugar and base of the nucleotide. Excision of the modified nucleobases of interest produces an abasic site (e.g., an apurinic or apyrimidinic, AP site) in the DNA target fragment at each position of the modified nucleobase of interest. In some instances, more than one DNA glycosylase or other enzyme(s) may be used to generate the abasic sites. The DNA glycosylase enzymes may also be engineered to inactivate functions not suitable to a desired outcome. For example, the lyase activity of an enzyme may be selectively inactivated, while glycosylase activity is maintained. Of note, the first complementary copy strands remain resistant to DNA glycosylase treatment, such the sites of their native nucleobases are not converted to abasic sites.

As used herein, the term “converted”, when used in reference to a DNA target fragment, refers to a DNA target fragment or a portion thereof which has been treated under conditions sufficient to excise the modified nucleobase of interest to generate abasic sites in an otherwise continuous polynucleotide strand. This process may also be referred to herein as “conversion of modified nucleobases to abasic sites”. In contrast to prior art methods of epigenetic detection that rely on chemical conversion of native nucleobases to differentiate between native and modified bases (e.g., bisulfite conversion of native cytosine), the methods of the present invention provide advantages of selective enzymatic excision of modified nucleobases, while native nucleobases are not altered. Thus, overall damage to the DNA targets fragments is not as widespread and the complexity of the genetic code is not as dramatically reduced relative to methods based on bisulfite conversion.

The method may then include Step F of denaturing the sample of double stranded DNA fragments to release converted parental DNA template strands 105a and 105b. As described, in some instances, the DNA templates are immobilized on a solid support prior to the first primer extension reaction to enable separation from the first complementary copy strands, which partition into solution following denaturation. In other instances, the first complementary copy strands are retained on a solid support, enabling the DNA target fragments to partition into solution following denaturation. Following Step F, the DNA template strands and the first complementary copy strands are no longer coupled. As used herein, the term “coupled” is well-known to a person skilled in the art and refers to the process in which the two nucleic acid strands are held together. Coupling is achieved by the formation of hydrogen bonds, e.g., between DNA template strands and their complementary copy strands. As such, in the context of the present disclosure, the terms “hybridized” and “hybridization” would fall under the definition of “coupled” and “coupling” respectively. I.e., for example, a complementary copy of a DNA template may be coupled to the template by hybridization.

The method may then include Step G of performing a second primer extension reaction. The second primer extension reaction is directed by an extension oligonucleotide hybridized to, e.g., a region in the adapter sequence of the DNA templates using a second DNA polymerase to produce second complementary copies 109a and 109b of the DNA target strand templates. The second DNA polymerase is selected for its ability to synthesize a complementary copy strand past (e.g., through and beyond) the positions of the abasic sites in the target fragment template. DNA polymerases exhibiting this property may be referred to as “bypass polymerases” and may include translesion DNA polymerases. As discussed with reference to Step D, in certain embodiments, either one of the DNA template strand or the second complementary strand may be selectively immobilized on a solid support to enable purification of the second complementary strand from the template strand.

According to the present invention, the nucleobases incorporated in the daughter strand at positions opposite abasic sites in the parental template do not form canonical Watson-Crick base pairs with the original, unconverted nucleobase under the extension conditions used in this step. In the example illustrated in FIG. 1C, the nucleotide incorporated opposite the abasic sites in the template strand is identified as “not G”, as G would normally base pair with 5-mC, the converted nucleobase of interest in this case. Thus, “not G” is any nucleobase other than G, e.g., any one of adenine (A), cytosine (C), or thymine (T).

In some instances, the second DNA polymerase may be selected based on its substrate specificity and incorporation of a preferred nucleotide at positions opposite the abasic sites in the converted template strand. For example, a DNA polymerase with a known preference for incorporating dATP opposite abasic sites in the template would be suitable for the detection of modified cytosine in the target fragment, as “A” does not normally base pair with “C”. Several DNA polymerases are known in the art to exhibit specific preferences for nucleotide incorporation at abasic sites, as discussed further herein.

The methods may then include Step H of determining the nucleotide sequence of the first and second complementary copy strands. Diverse sequencing platforms and methodologies are suitable for the practice of the present invention. In one example, the sequencing method is the nanopore-based “Sequencing by Expansion” (SBX®), see, e.g., Applicant's U.S. Pat. Nos. 7,939,259 and 10,301,345 and Published Application No.s, WO2020/172,479 and WO2020/236,526, which are herein incorporated by reference in their entireties).

The methods may then include Step I of comparing the sequence reads of the first and second complementary copy strands to identify the positions of the modified nucleobase of interest in the original DNA target fragment (e.g., using art-recognized bioinformatic analysis tools). The first complementary strand is used as a reference sequence, as it encodes the genetic information of the DNA target fragment. In contrast, the second complementary strand encodes the epigenetic information of the DNA target fragment. Differences in the sequences of the first and second complementary copy strands at a specific position (e.g., a base substitution) indicate the position of the modified nucleobase of interest in the sequence of the DNA target fragment. In the example depicted in FIG. 1D, “not G” detected in the second complementary strand at the same position as “G” in the first complementary strand indicates that the DNA target fragment originally included a 5-mC residue at this position in the opposite strand.

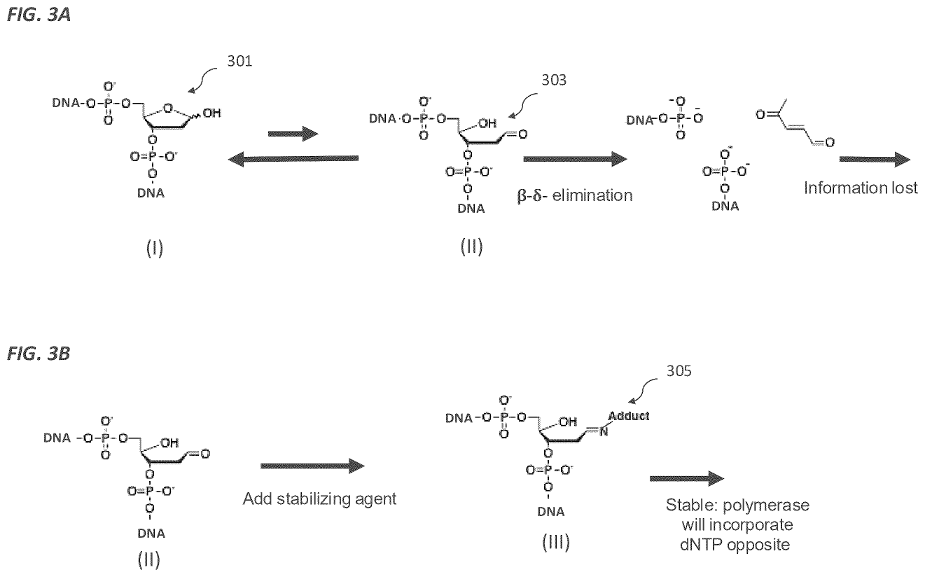

In some instances, the methods of the present invention may include additional steps to stabilize the abasic sites generated in the converted DNA templates prior to generating the second complementary copies (Step G). As summarized in the illustration in FIG. 3A, it is known in the art that abasic sites in DNA exist as an equilibrating mixture of two structural forms: (I) a closed-ring hemi acetal, 301 and (II) an open-ring aldehyde alcohol, 303. The open-ring aldehyde 303 is a highly reactive compound. Accordingly, abasic residues in DNA fragments convert into strand breaks via a β-elimination reaction in which the 3′ phosphodiester bond of the ring-opened aldehyde form is hydrolyzed to generate a 3′-terminal unsaturated sugar and a terminal 5′-phosphate. The presence of nucleophilic molecules, including thiols, amines, polyamines, and basic proteins in the environment, further favors this undesirable reaction. As is readily apparent to one of skill in the art, strand breaks are detrimental in that they prevent replication of the target fragment and result in the loss of information.

To overcome this problem, in certain embodiments, the methods disclosed herein may include use of stabilizing agents that prevent chemical degradation of the open-ring aldehyde 303 and the subsequent strand breakage. As depicted in FIG. 3B, in one instance, the stabilizing agent may be a chemical that covalently reacts with the abasic site to form stable adduct 305. As used herein, the term “adduct” refers to a product of a direct covalent addition of two or more distinct molecules, resulting in a single reaction product containing all atoms of all components and is thus a distinct molecular species. In other instances, the stabilizing agent may be a soluble buffer additive or other physicochemical reaction condition that does not covalently react with the abasic sites.

Further details regarding the methods and embodiments described above are provided below.

The practice of the present invention will employ, unless otherwise indicated, conventional techniques of molecular biology, microbiology, recombinant DNA, and so forth which are within the skill of the art. Such techniques are explained fully in the literature. See e.g., Sambrook, Fritsch, and Maniatis, MOLECULAR CLONING: A LABORATORY MANUAL, Second Edition (1989), OLIGONUCLEOTIDE SYNTHESIS (M. J. Gait Ed., 1984), the series METHODS IN ENZYMOLOGY (Academic Press, Inc.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987).

### DNA Sample/DNA Target Fragments

In one aspect, DNA from a biological sample is obtained or provided. The DNA obtained or provided from the biological sample may be genomic DNA, mitochondrial DNA, cell-free DNA (cfDNA), circulating tumor DNA (ctDNA), or a combination thereof.

DNA samples may be obtained from a patient or subject, from an environmental sample, or from an organism of interest. In embodiments, the DNA sample is extracted, purified, or derived from a cell or collection of cells, a body fluid, a tissue sample, an organ, and/or an organelle. In some embodiments, the sample DNA is whole genomic DNA.

In some instances, genomic DNA and mitochondrial DNA may be obtained separately from the same biological sample or source. Many different methods and technologies are available for the isolation of genomic DNA and mitochondrial DNA. In general, such methods involve disruption and lysis of the starting material followed by the removal of proteins and other contaminants and finally recovery of the DNA. Removal of proteins can be achieved, for example, by digestion with proteinase K, followed by salting-out, organic extraction, gradient separation, or binding of the DNA to a solid-phase support (either anion-exchange or silica technology). Mitochondrial DNA may be isolated similarly following initial isolation of mitochondria. DNA may be recovered by precipitation using ethanol or isopropanol. There are also commercial kits available for the isolation of nuclear or mitochondrial DNA. The choice of a method depends on many factors including, for example, the amount of sample, the required quantity and molecular weight of the DNA, the purity required for downstream applications, and the time and expense.

The methods of the present disclosure, in certain embodiments, utilize mild enzymatic and chemical reactions that avoid the substantial degradation associated with methods like bisulfite sequencing. Thus, the methods are useful in analysis of low-input samples, such as circulating cell-free DNA, circulating tumor DNA, and in single-cell analysis.

In some embodiments, the DNA sample is circulating cell-free DNA (cfDNA), which is DNA found in the blood and is not present within a cell. cfDNA can be isolated from blood or plasma using methods known in the art. Commercial kits are available for isolation of cfDNA including, for example, the Circulating DNA Kit (Qiagen). The DNA sample may result from an enrichment step, including, but is not limited to antibody immunoprecipitation, chromatin immunoprecipitation, restriction enzyme digestion-based enrichment, hybridization-based enrichment, or chemical labeling-based enrichment.

In some instances, the isolated DNA is fragmented into a plurality of shorter double stranded DNA target fragments. In general, fragmentation of DNA may be performed physically, or enzymatically.

For example, physical fragmentation may be performed by acoustic shearing, sonication, microwave irradiation, or hydrodynamic shear. Acoustic shearing and sonication are the main physical methods used to shear DNA. For example, the Covaris® instrument (Woburn, MA) is an acoustic device for breaking DNA into 100 bp-5 kb. Covaris also manufactures tubes (gTubes) which will process samples in the 6-20 kb for Mate-Pair libraries. Another example is the Bioruptor® (Denville, NJ), a sonication device utilized for shearing chromatin, DNA and disrupting tissues. Small volumes of DNA can be sheared to 150 bp-1 kb in length. The Hydroshear® from Digilab (Marlborough, MA) is another example and utilizes hydrodynamic forces to shear DNA. Nebulizers, such as those manufactured by Life Technologies (Grand Island, NY) can also be used to atomize liquid using compressed air, shearing DNA into 100 bp-3 kb fragments in seconds. As nebulization may result in loss of sample, in some instances, it may not be a desirable fragmentation method for limited quantities samples. Sonication and acoustic shearing may be better fragmentation methods for smaller sample volumes because the entire amount of DNA from a sample may be retained more efficiently. Other physical fragmentation devices and methods that are known or developed can also be used.

Various enzymatic methods may also be used to fragment DNA. For example, DNA may be treated with DNase I, or a combination of maltose binding protein (MBP)-T7 Endo I and a non-specific nuclease such as Vibrio vulnificus nuclease (Vvn). The combination of non-specific nuclease and T7 Endo synergistically work to produce non-specific nicks and counter nicks, generating fragments that disassociate 8 nucleotides or less from the nick site. In another example, DNA may be treated with NEBNext® dsDNA Fragmentase® (NEB, Ipswich, MA). NEBNext® dsDNA Fragmentase generates dsDNA breaks in a time-dependent manner to yield 50-1,000 bp DNA fragments depending on reaction time. NEBNext dsDNA Fragmentase contains two enzymes, one randomly generates nicks on dsDNA and the other recognizes the nicked site and cuts the opposite DNA strand across from the nick, producing dsDNA breaks. The resulting DNA fragments contain short overhangs, 5′-phosphates, and 3′-hydroxyl groups.

In some instances, the DNA sample is fragmented into specific size ranges of target fragments. For example, the DNA sample may be fragmented into fragments in the range of about 25-100 bp, about 25-150 bp, about 50-200 bp, about 25-200 bp, about 50-250 bp, about 25-250 bp, about 50-300 bp, about 25-300 bp, about 50-500 bp, about 25-500 bp, about 150-250 bp, about 100-500 bp, about 200-800 bp, about 500-1300 bp, about 750-2500 bp, about 1000-2800 bp, about 500-3000 bp, about 800-5000 bp, or any other size range within these ranges. For example, the DNA sample may be fragmented into fragments of about 50-250 bp. In some instances, the fragments may be larger or smaller by about 25 bp.

The DNA target fragments may be any DNA fragment, derived from a biological sample, having a sequence of interest that may or may not include epigenetic modifications or DNA damage to one or more nucleobases. In some aspects, the DNA target fragments may include cytosine modifications (i.e., 5-mC, 5-hmC, 5-fC, and/or 5-caC). The DNA target fragments can be a single DNA molecule in the sample, or may be the entire population of DNA molecules in a sample (or a subset thereof) having, e.g., a cytosine modification. The DNA target fragments can comprise a plurality of DNA sequences such that the methods described herein may be used to generate a library of DNA target fragments that can be analyzed individually (e.g., by determining the sequence of individual targets) or in a group (e.g., by multiplexed DNA sequencing methodologies).

In embodiments, the methods described herein include the step of adding adapter DNA molecules to double stranded DNA target fragments. An adapter DNA, or DNA linker, is a short, chemically-synthesized, single- or double-stranded oligonucleotide that can be ligated to one or both ends of other DNA molecules. Double-stranded adapters can be synthesized so that each end of the adapter has a blunt end or a 5′ or 3′ overhang (i.e., sticky ends). DNA adapters are ligated to the DNA target fragments to provide sequences for, e.g., primer extension reactions and sequencing reactions with complimentary primers and/or for bioinformatic analysis (e.g., clustering of related sequences into families based on shared unique molecular identifier barcodes, UMIs).

Prior to ligation of adapters, the ends of the DNA fragments can be prepared for ligation. For example, by end repair and creating blunt ends with 5′ phosphate groups. Fragmented DNA may be rendered blunt-ended by a number of methods known to those skilled in the art. In a particular method, the ends of the fragmented DNA are “polished” with T4 DNA polymerase and Klenow polymerase, a procedure well known to skilled practitioners, and then phosphorylated with a polynucleotide kinase enzyme. A single ‘A’ deoxynucleotide is then added to both 3′ ends of the DNA molecules using Taq polymerase or Klenow exo minus polymerase enzyme, producing a one-base 3′ overhang that is complementary to the one-base 3′ ‘T’ overhang on the double-stranded end of an adaptor.

In some instances, the adapters may include two oligonucleotides that are partially complementary such that they hybridize to form a region of double stranded sequence, but also retain a region of single stranded, non-hybridized sequence. The region of single stranded sequence may include “universal” oligonucleotide binding sequences, enabling all target fragments in a library to bind to the same oligonucleotide, which may be a capture oligonucleotide, to localize target fragments to a solid-support, an oligonucleotide primer for a primer extension reaction, a PCR primer, sequencing primer, or combinations thereof. In certain instances, the adapters may include two regions of single-stranded, non-hybridized sequence (i.e., a first, 5′ single stranded region and a second, 3′ single stranded region). This configuration is known in the art as a “Y” adapter. The first and second single stranded regions of a Y adapter are not complementary and may include different primer hybridization sequences and other features.

The portions of the two single stranded regions of the adapters typically include at least 10, or at least 15, or at least 20 consecutive nucleotides on each strand. The lower limit on the length of the single stranded regions will typically be determined by function, for example, the need to provide a suitable sequence for binding of a primer for primer extension, PCR and/or sequencing. Theoretically there is no upper limit on the length of the single stranded regions, except that in general it is advantageous to minimize the overall length of the adapter, for example, in order to facilitate separation of unbound adapters from adapter-ligated double stranded DNA target fragments following the ligation step. Therefore, it is preferred that the single stranded regions should be fewer than 50, or fewer than 40, or fewer than 30, or fewer than 25 consecutive nucleotides in length on each strand.

The double stranded regions of the adapter is a short double stranded region, typically comprising 5 or more consecutive base pairs, formed by annealing of the two partially complementary polynucleotide strands. Generally, it is advantageous for the double stranded region to be as short as possible without loss of function. By “function” in this context is meant that the double stranded region forms a stable duplex under standard reaction conditions for the enzyme-catalyzed nucleic acid ligation reaction.

The precise nucleotide sequence of the adapters is generally not material to the invention and may be selected by the user such that the desired sequence elements are ultimately included in the common sequences of the library of adapter-ligated double stranded DNA target fragments. Additional sequence elements may be included, for example, to provide binding sites for primers which will ultimately be used in sequencing of complementary copy strands of the DNA target fragments. The adapters may further include “tag” sequences, unique molecular identifiers (UMI), and/or sample identifier sequences, which can be used to tag, track, and differentiate target fragments and complementary copies thereof derived from a particular source. The general features and use of such sequences is well known in the art.

The ends of the single stranded regions of the adapters may be biotinylated or bear another functionalities that enables it to be captured, or immobilized, on a surface, such as a solid support. Alternative functionalities other than biotin are known in the art, e.g., as described in Applicant's published Patent Application no. WO2020/172479 entitled, “Methods and Devices for Solid-Phase Synthesis of Xpandomers for use in Single Molecule Sequencing”, which is herein incorporated by reference in its entirety.

“Ligation” of adapters to the 5′ and 3′ ends of each fragmented double stranded nucleic acid target fragment involves joining of the two polynucleotide strands of the adapter to the double-stranded target polynucleotide such that covalent linkages are formed between both strands of the two double-stranded molecules. Preferably such covalent linking takes place by formation of a phosphodiester linkage between the two polynucleotide strands but other means of covalent linkage (e.g., non-phosphodiester backbone linkages) may be used. However, it is an essential requirement that the covalent linkages formed in the ligation reactions allow for read-through of a polymerase, such that the resultant construct can be copied in a primer extension reaction using primers which bind to sequences in the regions of the adapter-target construct that are derived from the adapter molecules.

In some instances, the adapters and DNA target fragments may be incubated with a ligase to covalently link the adapters and DNA target fragments. Ligase catalyzes the formation of a phosphodiester bond between juxtaposed 5′ phosphate and 3′ hydroxyl termini in duplex DNA or RNA. The enzyme will join blunt end and cohesive end termini as well as repair single stranded nicks in duplex DNA. An exemplary ligase is T4 ligase, which is the most frequently used enzyme for cloning. Another ligase that may be used is E. coli DNA ligase, which preferentially connects cohesive double-stranded DNA end but is also active on blunt ends DNA in the presence of Ficoll or polyethylene glycol. Another ligase that may be used is DNA ligase Ilia, which is known to function in mitochondria.

The products of the ligation reaction may be subjected to purification steps in order to remove unbound adapter molecules before the adapter-target constructs are processed further.

The ligation of adapters to both free ends of the double stranded DNA target fragments gives rise to a pool of adapter-ligated double stranded DNA target fragments with adapters at the 5′ and 3′ ends of the target.

There are several standard methods for separating the strands of an adapter-ligated double stranded DNA target fragment by denaturation, including thermal denaturation, or chemical denaturation in either 100 mM sodium hydroxide solution or formamide solution. The pH of a solution of single-stranded DNA fragments can be neutralized by adjusting with an appropriate solution of acid, or preferably by buffer-exchange through a size-exclusion chromatography column pre-equilibrated in a buffered solution.

### First Complementary Copy Strand

In embodiments disclosed herein, a single stranded DNA target fragment (i.e., a parent strand) provides a template for the synthesis of a first complementary copy of the target fragment (i.e., a first daughter strand) via a primer extension reaction. The term “primer extension reaction” is used herein interchangeably with the term “nucleic acid polymerization reaction” and refers to an in vitro method for making a new strand of nucleic acid or elongating an existing nucleic acid in a template-dependent manner. The first complementary copy strand is synthesized by extending an oligonucleotide primer with a first DNA polymerase, such that a first complementary copy of the template strand is extended in the 3′ direction of the oligonucleotide primer.

In embodiments where the DNA target fragment is double stranded, one or both strands may serve as the template for the primer extension reactions. For example, where one strand (the “sense” strand) serves as template, a complementary copy is generated, which is complementary to the sense strand. Likewise, where the antisense strand serves as template, a complementary copy is generated, which is complementary to the antisense strand. Where both strands serve as template, a separate complementary copy is generated for each of the sense and antisense strands. In a preferred embodiment, each strand of a double stranded DNA target fragment is a template nucleic acid.

As used herein, the term “complementary” refers to nucleic acid sequences that are capable of forming Watson-Crick base-pairs. For example, a complementary sequence of a first sequence is a sequence which is capable of forming Watson-Crick base-pairs with the first sequence. The term “complementary” does not necessarily mean that a sequence is complementary to the full-length of its complementary strand, but the term can mean that the sequence is complementary to a portion thereof. Thus, in some embodiments, complementarity encompasses sequences that are complementary along the entire length of the sequence or a portion thereof. For example, two sequences can be complementary to each other along at least 70%, 75%, 80%, 85%, 90%, 91%, 92%, 93%, 94%, 95%, 96%, 97%, 98%, 99%, or 100% of the length of the sequence. Here, the term “sequence” encompasses, but is not limited to, nucleic acid sequences, polynucleotides, oligonucleotides, probes, primers, primer-specific regions, and target-specific regions. Despite any mismatches, the two sequences should have the ability to selectively hybridize to one another under appropriate conditions.

Primer extension can be performed by any method that allows for polymerase-based extension of a primer annealed (i.e., hybridized) to the single stranded DNA target fragment. In some embodiments, simple primer extension involves addition of a primer and a first DNA polymerase to the target DNA fragment under conditions to allow for primer hybridization and primer extension by the polymerase. Of course, such a reaction includes the necessary nucleotides, buffers, and other reagents known in the art for primer extension. Importantly, the nucleotides included in the primer extension reaction are “native”, i.e., unmodified, nucleotides and, thus, the first complementary copy strand will not include modifications to the nucleobase of interest. The first complementary copy strand is generated to encode and preserve the genetic sequence of the DNA target strand.

Any number of methods are known for detecting primer extension products. In some embodiments, the primer is detectably labeled (e.g., at its 5′ end or otherwise located to not interfere with 3′ extension of the primer) and following primer extension, the length and/or quantity of the labeled extension product is detected by detecting the label.

In a particular embodiment, the primer used in the primer extension reaction anneals to a primer-binding sequence (in one strand) in a single stranded region of the adapter. The term “annealing” as used in this context refers to sequence-specific binding/hybridization of the primer to a primer-binding sequence in an adapter region of the adapter-ligated DNA target fragment under the conditions used for the primer annealing step of the initial primer extension reaction. Primer annealing conditions are well known in the art (see, e.g., Sambrook et al., 2001, Molecular Cloning, A Laboratory Manual, 3rd Ed, Cold Spring Harbor Laboratory Press, Cold Spring Harbor Laboratory Press, NY; Current Protocols, eds Ausubel et al.).

In preferred embodiments, the first DNA polymerase is a high-fidelity DNA polymerase. The fidelity of a DNA polymerase is the result of accurate replication of a desired template. Specifically, this involves multiple steps, including the ability to read a template strand, select the appropriate nucleoside triphosphate and insert the correct nucleotide at the 3′primer terminus, such that Watson-Crick base pairing is maintained. In addition to effective discrimination of correct versus incorrect nucleotide incorporation, some DNA polymerases possess a 3′→5′ exonuclease activity. This activity, known as “proofreading”, is used to excise incorrectly incorporated mononucleotides that are then replaced with the correct nucleotide.

In certain embodiments, suitable high-fidelity DNA polymerases for the practice of the present invention include KAPA HiFi DNA Polymerase, commercially available from Roche Diagnostics Corp., Q5® High-Fidelity DNA Polymerase, commercially available from New England Biolabs, Inc., and an engineered Pfu DNA polymerase, such as Pfu-X, commercially available from Jena Biosciences.

### Solid-Phase Synthesis

In certain embodiments, the first primer extension reaction may be conducted on a solid support. Thus, in further aspects, the invention provides a method for solid-phase nucleic acid synthesis using adapter-ligated DNA target fragments, which have known sequences at their 5′ and 3′ ends (e.g., sequence features that have been designed into the adapters).

The terms “solid support”, “solid-state”, “solid-phase”, and “substrate” are used herein interchangeably and refer to a material or group of materials having a rigid or semi-rigid surface or surfaces. In many embodiments, at least one surface of the solid support will be substantially flat, e.g., a surface of a polymeric microfluidic card or chip. In some embodiments it may be desirable to physically separate regions of a card or chip for different reactions with, for example, etched channels, trenches, wells, raised regions, pins, or the like. According to other embodiments, the solid support(s) will take the form of insoluble beads, resins, gels, membranes, microspheres, or other geometric configurations composed of, e.g., controlled pore glass (CPG) and/or polystyrene.

The invention encompasses solid-phase synthesis methods in which a capture moiety is immobilized on a solid support. In certain instances, the capture moiety includes a first end covalently bound to the solid support and a second end that provides a functional group capable of binding to the 5′ end of a single stranded adapter-ligated DNA target fragment. In this case, the single stranded DNA target fragment is immobilized on the solid support, while the complementary copy strand is not immobilized on the solid support. In other instances, the capture moiety includes an extension oligonucleotide that is capable of hybridizing to the 3′ end of the single stranded adapter-ligated target fragment. The single stranded adapter-ligated DNA target fragment is hybridized to the extension oligonucleotide and a primer extension reaction is carried out. In this case, only the complementary copy strand is immobilized on the solid support. These alternative solid-phase synthesis configurations are illustrated in FIG. 2A and FIG. 2B.

The term “immobilized”, as used herein, refers to the association, attachment, or binding between a molecule (e.g., linker, adapter, or oligonucleotide) and a support in a manner that provides a stable association under the conditions of elongation, amplification, ligation, and other processes as described herein. Such binding can be covalent or non-covalent. Non-covalent binding includes electrostatic, hydrophilic and hydrophobic interactions. Covalent binding is the formation of covalent bonds that are characterized by sharing of pairs of electrons between atoms. Such covalent binding can be directly between the molecule and the support or can be formed by a cross linker or by inclusion of a specific reactive group on either the support or the molecule or both. Covalent attachment of a molecule can be achieved using a binding partner, such as avidin or streptavidin, immobilized to the support and the non-covalent binding of the biotinylated molecule to the avidin or streptavidin. Immobilization may also involve a combination of covalent and non-covalent interactions.

Any suitable covalent attachment means known in the art may be used for these purposes. The chosen attachment chemistry will depend on the nature of the solid support and any derivatization or functionalities applied thereto. The extension oligonucleotide may include a moiety, which may be a non-nucleotide chemical modification, to facilitate attachment. Certain exemplary embodiments of suitable surface chemistries include conventional streptavidin/biotin interaction chemistry and involve functionalization of a solid support, e.g., with a linker moiety that includes terminal a biotin moiety. In this embodiment, the 5′ end of single stranded DNA fragment (or oligonucleotide) is bound to the linker moiety. Attachment is mediated by a streptavidin moiety provided by the 5′ end of the single stranded DNA fragment. The linker moieties disclosed herein may be of sufficient length to connect the single stranded DNA fragment to the support such that the support does not significantly interfere with primer extension reaction.

Alternatively, immobilization of a capture moiety or oligonucleotide (e.g., an extension oligonucleotide) to a solid support may be accomplished by covalent linkage of the capture oligonucleotide to the solid support via a click reaction. In this embodiment, the covalent linkage may be mediated by a maleimide-PEG-alkyne linker that is crosslinked to the solid support. An alkyne moiety provided by the end of the linker distal to the substrate is capable of reacting with an azide group provided by the 5′ end of the capture oligonucleotide. Methods of functionalizing a solid support with maleimide-linker polymers is provided in Applicant's published Patent Application No. WO2020/172479, which is herein incorporated by reference in its entirety.

In certain instances, the linkage between the capture moiety and the solid support is cleavable, enabling primer extension products to be released from the support following synthesis. Cleavable linkers and methods of cleaving such linkers are known and can be employed in the provided methods using the knowledge of those of skill in the art. For example, the cleavable linker can be cleaved by an enzyme, a catalyst, a chemical compound, temperature, electromagnetic radiation or light. Optionally, the cleavable linker includes a moiety hydrolysable by beta-elimination, a moiety cleavable by acid hydrolysis, an enzymatically cleavable moiety, or a photo-cleavable moiety. In some embodiments, a suitable cleavable moiety is a photocleavable (PC) spacer or linker phosphoramidite available from Glen Research.

### Glycosylase-Mediated Excision of Modified Nucleobases

In one aspect, the methods of the present invention include the step of treating the double stranded DNA products of the first primer extension reaction with a DNA glycosylase enzyme to specifically excise the modified base of interest. Many DNA glycosylases are known in the art, targeting a wide range of specifically modified nucleobases and DNA damage elements, including sequence mismatches and a large range of epigenetic modifications. Exemplary epigenetic modifications detectable by the described methods include, but are not limited to, 5-methylcytosine (5-mC), 5-hydroxymethylcytosine (5-hmC), 5-carboxycytosine (5-caC), f5-ormylcytosine (5-fC), 8-oxo-7,8-dihyroguanine (oxoG), uracil, methyladenine (mA), and others.

There are two main classes of DNA glycosylases: monofunctional and bifunctional. Monofunctional glycosylases have only glycosylase activity and cleave the N-glycosidic bond linking a damaged or modified nucleobase to the sugar-phosphate backbone of DNA. All DNA glycosylases cleave glycosidic bonds, but differ in their base substrate specificity and in their reaction mechanisms, Bifunctional glycosylases also possess apurinic or apyrimidinic site (AP) lyase activity that enables them to cut the phosphodiester bond of DNA at a base lesion, creating a single-strand break.

A non-limiting list of exemplary DNA glycosylases that are useful in the methods of the present invention are set forth in Table 1. In some instances, one or more of the DNA glycosylases listed in Table 1 may be used in the described methods to excise modified bases of interest from DNA target fragments. While select DNA glycosylases are specifically identified in this disclosure, it is understood that any suitable DNA glycosylase can be used in the performing the base excision step of the described methods.

TABLE 1
DNA Glycosylases

| Enzyme | Type | Species | Substrate(s) |
|---|---|---|---|
| N-methylpurine | Monofunctional | Human | 3-mA; 7-mG; 3-mG |
| DNA Glycosylase | DNA glycosylase | | |
| (MPG) | | | |
| MutY Homolog | Monofunctional | Human | A mis-paired with 8-oxoG, C, |
| (MUTYH) | DNA glycosylase | | or G |
| Nth-like DNA | Bifunctional DNA | Human | Thymine glycol (Tg); 2,6- |
| Glycosylase 1 | Glycosylase | | diamino-4-oxo-5- |
| (NTHL1) | | | formamidopyrimidine (FapyG); |
| | | | 5-hmC; 5-hydroxyuracil (5- |
| | | | HU) |
| Nei-like DNA | Bifunctional DNA | Human | Tg; FapyG; 4,6-diamino-5- |
| Glycosylase 1 | Glycosylase | | formamidopyrimidine (FapyA); |
| (NEIL1) | | | 8-oxoG; 5-HU |
| NEIL2 | Bifunctional DNA | Human | Tg; FapyG; FapyA; 8-oxoG; 5- |
| | Glycosylase | | HU |
| NEIL3 | Mono- or | Human | FapyG; FapyA |
| | bifunctional DNA | | |
| | glycosylase | | |
| 8-oxoguanine DNA | Mono- or | Human | 8-oxoG |
| glycosylase (OGG1) | bifunctional DNA | | |
| | glycosylase | | |
| Uracil DNA | Monofunctional | Human | U; 5-FU |
| Glycosylase 1 | DNA glycosylase | | |
| (Ung1) | | | |
| Uracil DNA | Monofunctional | Human | U, 5-FU |
| Glycosylase 2 | DNA glycosylase | | |
| (Ung2) | | | |
| Single-strand | Monofunctional | Human | 5-hmU; U mis-paired with G; |
| selective | DNA glycosylase | | 5-FU |
| monofunctional | | | |
| uracil glycosylase | | | |
| (SMUG1) | | | |
| Thymine DNA | Monofunctional | Human | U or T mis-paired with G; 5- |
| Glycosylase (TDG) | DNA glycosylase | | hmU; 5-fC, 5-CaC |
| Methyl binding | Monofunctional | Human | U or T mis-paired with G; 5- |
| domain 4 (MBD4) | DNA glycosylase | | hmU in CpG islands |
| Fpg | Bifunctional DNA | E. coli | 8-oxoG; 5-caC |
| | Glycosylase | | |
| Ung | Monofunctional | E. coli | U |
| | DNA glycosylase | | |
| Demeter (DME) | Bifunctional DNA | A. thaliana | 5-mC |
| | Glycosylase | | |
| ROS1 | Bifunctional DNA | A. thaliana | 5-mC; T mis-paired with G |
| | Glycosylase | | |

In one embodiment, the present methods utilize a DNA glycosylase that acts directly on 5-mC, i.e., a glycosylase that is capable of hydrolyzing the glycosidic bond between the 5-mC residue and the sugar-phosphate backbone. For example, a suitable DNA glycosylase that directly excises 5-mC may be a member of the DEMETER (DME) family of DNA glycosylases, e.g., DME, ROS1, or DMEL. The DME gene of Arabidopsis encodes a 1,729 amino acid protein with a centrally located DNA glycosylase domain (amino acids 1167-1368) that includes a helix-hairpin-helix (HhH) motif. The HhH motif in DME catalyzes excision of 5-mC (see, e.g., Choi et al., 2002. Cell 110:33-42). In certain embodiments, the DME glycosylase may be a variant that comprises amino acids 1167-1368 but lacks certain other regions ofthe protein.

In some instances, a suitable DNA glycosylase that acts directly on 5-mC may be an orthologue of DME. As used herein, the term “orthologue” means one of two or more homologous gene sequences found in different species. Table 2 sets forth an exemplary list of DME orthologues that may be used according to the present invention.

TABLE 2
DME Orthologues

| Species | Accession number/comments |
|---|---|
| Bathycoccus prasinos | XP_007509798 |
| Ostreococcus tauri | CEF97905 |
| Coccomyxa subellipsoidea | XP_00549215 |
| volvox carteri | XP_002950363 |
| Micromonas commode | XP_002500051 |
| Auxenochlorella protothecoides | XP_011399155 |
| Gonium pectoral | KXZ53817 |
| Physcomitrella patens | XP_001785533 |
| Physcomitrella patens | XP_001777735 |
| Nannochloropsis gaditana | EWM29049 |
| Klebsormidium nitens | GAQ84186 |
| Selaginella moellendorffii | XP_002990766 |
| Marchantia polymorpha | OAE23247 |
| N. gaditana | EJK65312 |
| Prunus avium | XP_021824383 |
| Prunus avium | XP_021833615 |
| Arabidopsis thalian | OAO92434 (DME) |
| Capsicum annuum | XP_016550501 |
| Pyrus × bretschneideri | XP_009353006 |
| Capsicum annuum | XP_016543466 |
| Capsicum annuum | XP_016542890 |
| Arabidopsis thaliana | OAP06165 (DML2) |
| Arabidopsis thaliana | OAP10755 (ROS1) |
| Rubus occidentalis | GDR |
| Pyrus × bretschneideri | XP_009366838 |
| Malus domestica | XP_017178103 |
| Capsicum annuum | XP_016554458 |
| Capsicum annuum | Capsicum annuum_XP_016562450 |
| Arabidopsis thaliana | OAO99112 (DML3) |
| Capsicum annuum | XP_016566581 |
| Arabidopsis thaliana | NP_973767 (EndIII2) |
| Arabidopsis thaliana | NP_565725 (EndIII) |
| Arabidopsis thaliana | NP_566893 (DNAglycosylase protein) |
| Capsicum annuum | XP_016563498 |

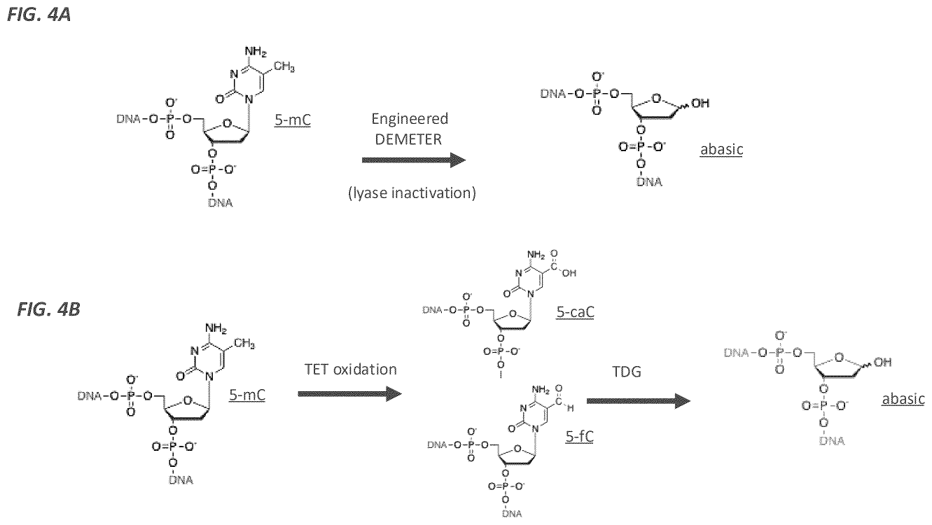

In instances where the DNA glycosylase is a bifunctional enzyme, the glycosylase (e.g., DME, or an orthologue thereof), may be mutated to inactivate lyase activity, while still retaining glycosylase activity, as depicted in FIG. 4A. The reaction mechanism of bifunctional DNA glycosylases is well known in the art (see, e.g, Scharer and Jiricny. 2001. Bioessays 23: 270-281). In some cases, a conserved aspartic acid acquires a proton from a conserved lysine residue that attacks the C1′ carbon of the deoxyribose ring, creating a covalent DNA-enzyme intermediate. Beta or gamma elimination reactions release the enzyme from the DNA and cleave one of the phosphodiester bonds. Mutant forms of DME in which the invariant aspartic acid at position 1304 or the lysine at position 1286 have been altered (e.g., variants D1304N or K1286Q) been shown to reduce DNA glycosylase activity while preserving enzyme structure and stability (see, e.g., Fromme et al. 2004 Nature 427: 652-656).

Other mutations that inactivate or optimize suitable features of the DNA glycosylase are also contemplated by the present invention. For example, the DNA glycosylase may be engineered to increase its stability and/or solubility. The DNA glycosylase may also be engineered to optimize for a desired substrate specificity.

In certain embodiments, thymine DNA glycosylase (TDG) may be used to excise its known targets, 5-carboxycytosine (5-caC) and 5-formylcytosine (5-fC). In further embodiments, as depicted in FIG. 4B, TDG may be used to identify 5-methylcytosine (5-mC) and 5-hydroxymethylcytosine (5-hmC), which are modified bases that it does not specifically recognize. For example, DNA target fragments may also be treated with a ten eleven translocation (TET) enzyme prior to treatment with TDG. The TET family proteins included three human proteins (TET1, TET2, and TET3) and are cytosine oxygenases that catalyze the conversion of 5-methylcytosine (5-mC) into 5-hydroxymethylcytosine (5-hmC). 5-hmC can be further oxidized into 5-formylcytosine (5-fC) and 5-carboxylcytosine (5-caC) by TET proteins (see, e.g., Parker, et. al. 2019. Biochemistry 58: 450-467). In another instance, a suitable TET enzyme may be any TET orthologue, e.g., ngTET, isolated from Naegleria (see, e.g., Hashimoto, et. al. 2014. Nature 506(7488): 391-395). Thus, in certain embodiments, TDG may be used to excise any existing 5-caC and 5-fC modified bases present in a DNA target fragment also treated with a TET enzyme.

Other comparable methods for altering the selective excision of modified bases are possible according to the present invention. For example, a similar method may be performed to detect the same bases discussed above using thymine DNA glycosylase (TDG) and uracil DNA glycosylase (UDG).

The base excision processes discussed herein may be performed using a purified enzyme, which may be a recombinant enzyme that includes a heterologous tag to facilitate purification. Protein tags are well known in the art and include, e.g., terminal poly-histidine tags that enable purification via immobilized metal affinity chromatography (IMAC). In certain instances, it may be desirable to include more than one protein purification step. For example, the glycosylases enzymes used in the methods disclosed herein should preferably be free of contaminating nucleic acids. In some instances, the protein purification step may include one or more of size-exclusion chromatography, ion exchange chromatography, affinity chromatography, heparin adsorption chromatography, and the like.

Of course, a nucleobase excision reaction will include a suitable buffer, cofactors, additives, and an amount of purified DNA glycosylase sufficient to achieve the desired base excision reaction such that the modified nucleobases of interest in a DNA target fragment are excised to generate abasic sites. An exemplary nucleobase excision reaction is described in Example 1.

Following treatment with the DNA glycosylase, the double stranded DNA fragment will be asymmetrically altered. Of note, the DNA template strand will lack a nucleobase at the positions of the original modified base of interest. In contrast, the first complementary copy strand remains unaltered (i.e., “unconverted”), as the native nucleobases incorporated during the first primer extension reaction will be resistant to glycosylation-mediated conversion to abasic sites.

### Stabilization of Abasic Sites in DNA Target Fragments

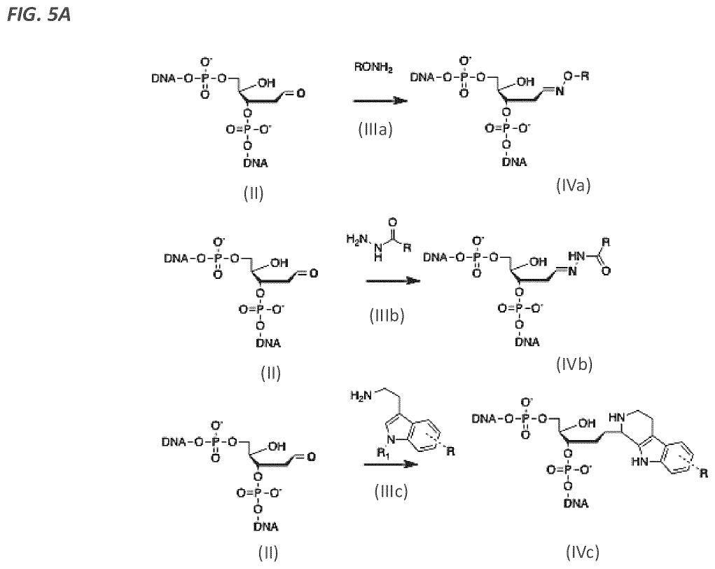

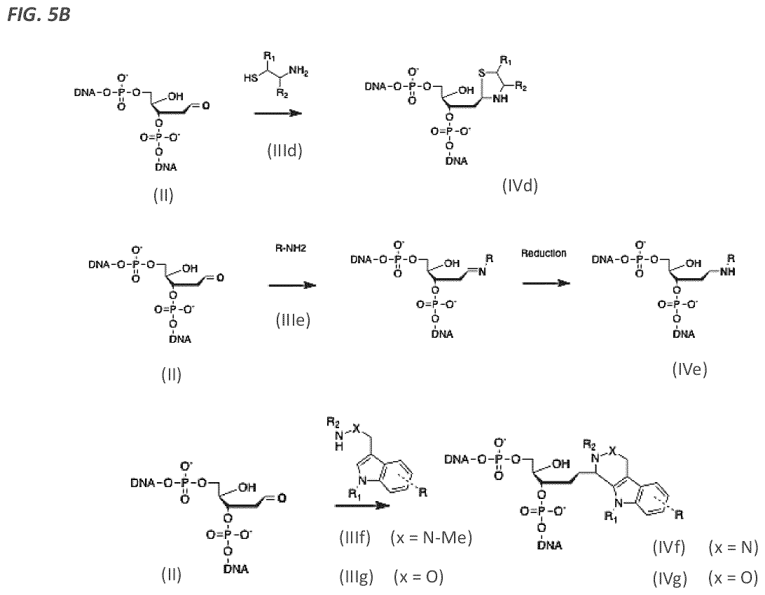

Advantageously, according to the methods of the present invention, abasic sites generated in DNA target fragments may be protected from further degradation with a stabilizing agent. In certain embodiments, a suitable stabilizing agent may be a chemical that covalently binds to the abasic site to form a stable abasic adduct. As discussed with reference to FIG. 3B, certain aldehyde-reactive compounds are known to react with the open-ring aldehyde form (II) of the abasic site to create stable open structures, that are referred to herein abasic adducts. Abasic adducts are refractory to enzymatic activity (e.g., lyase-mediated degradation) or to degradation-inducing chemical conditions, such as high pH. Some exemplary, non-limiting, structural classes of aldehyde-reactive stabilizing agents are illustrated in FIGS. 5A and 5B and described below. Each class varies in reaction rates, stability, and size of the resulting protected adduct product. The chemical properties of each abasic adduct product provide different chemoenzymatic properties with regard to duration of stabilization and suitability as a template for extension by a DNA polymerase.

As shown in FIG. 5A, in one embodiment, suitable stabilizing agents may be from the group of O-hydroxylamines (compound IIIa), which are a class of compounds known to react with the aldehydic group of the open-ring form of the abasic site (II) to create very stable oxime structures (compound IVa) that are refractory to β-elimination by enzymatic activity (e.g., AP or dRp lyases) or by high pH.

In another embodiment, suitable stabilizing agents may be from the group of acyl hydrazines (compound IIIb), which are a class of compounds that react with aldehydes (II) to form acyl hydrazones (compound IVb).

In another embodiment, suitable stabilizing agents may be from the group of tryptamines (compound IIIc), which reacts with aldehydes (II) via a Pictet-Spengler ring-forming reaction to form tricyclic heterocycles (compounds IVc).

As shown in FIG. 5B, In another embodiment, suitable stabilizing agents may be from the group of beta amino thiols (compound IIId) (e.g., cysteine), which are a class of compounds that react with aldehydes (II) to form cyclic thiazolidines (compound IVd).

In another embodiment, suitable stabilizing agents may be from the group of alkyl hydrazines (group IIIe), which are a class of compounds that react with aldehydes (II) to form alkyl hydrazones (compound IVe).

In another embodiment, suitable stabilizing agents may be from the group of hydrazino-iso-pictet-spengler indoles (compound IIIf), which reacts with abasic aldehydes (II) form to form tricyclic structures (compound IVf).

In another embodiment, suitable stabilizing agents may be from the group of methylaminooxy-iso-pictet-spengler indoles (group IIIg), which react with abasic aldehydes (II) to form tricyclic structures (compound IVg).

In other instances, the stabilizing agent may be an agent that does not covalently react with the abasic sites in DNA target fragments, e.g., a reaction additive or other physicochemical reaction condition. The following is a non-limiting list of exemplary stabilizing agents: 1. aqueous buffers lacking salt (e.g., water); 2. basic buffers at various concentrations (e.g., buffers based on ammonia, NaOH, or other hydroxides); 3. acidic buffers at various concentrations (e.g., buffers based on acetic acid, HCl or nitric acid); 4. urea; 5. detergents (e.g., SDS, Tween, or Triton); 6. solvents (e.g., acetonitrile, DMSO, formamide, DMF, or glycerol); 7. PEG and PEG variants; 8. guanidine salts; and 9. electric current or pulses of current applied to the reaction. In some instances, any suitable combination of the preceding stabilizing agents may be used.

In certain embodiments, the chemistries described herein may be used to form stable abasic adducts during treatment of DNA target fragments with one or more of a monofunctional DNA glycosylase, a bifunctional DNA glycosylase, or a bifunctional DNA glycosylase engineered to inactivate lyase activity.

In certain embodiments, the methods of the present invention may utilize a bifunctional DNA glycosylase to generate abasic sites that are stable and refractory to lyase-mediated backbone cleavage. In other words, the glycosylase activity may be uncoupled from the lyase activity of a bifunctional glycosylase, by chemically “knocking out” the latter. In some embodiments, this may be accomplished by including one or more of the abasic stabilizing agents disclosed herein in the glycosylase reaction. As discussed, the stabilizing agent forms a stable adduct at the abasic sites following excision of the modified nucleobase. Such abasic adducts are resistant to further lyase activity such that no strand excision occurs at these sites. This phenomenon is referred to herein as a biochemical knockout, or “hijack”, of DNA lyase activity.

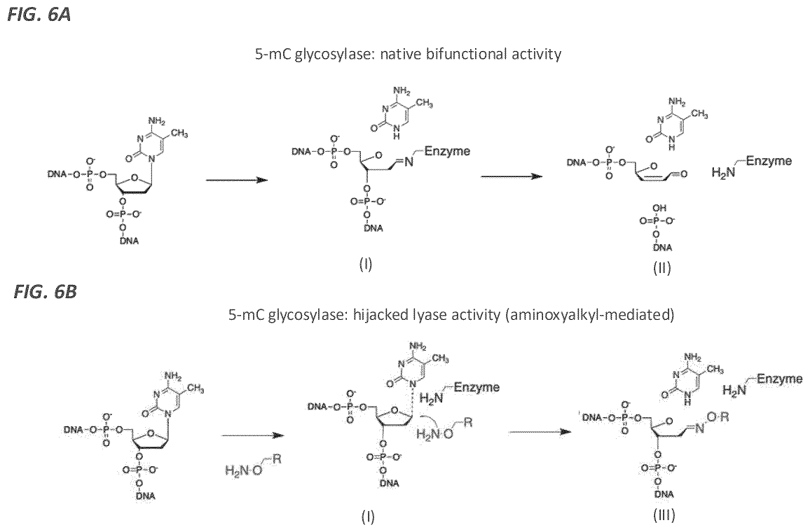

Biochemical hijack of DNA lyase activity is illustrated in simplified form in FIGS. 6A and 6B. FIG. 6A depicts the native activity of an exemplary bifunctional DNA glycosylase that acts on 5-mC (e.g., DEMETER). Following cleavage of the N-glycosidic bonds to release the methylated base, the enzyme forms a Schiff base intermediate (I) with the open-ring ribose moiety and proceeds to cleave the phosphodiester bond in the DNA backbone through a β-elimination reaction to produce a strand brake (II). FIG. 6B depicts knockout of lyase activity with an aminoxyalkyl compound. As used herein, the term “aminoxyalkyl” is used to denote a structure that is an O-alkylated derivative of hydroxylamine and has the general formula of H₂N—O—R where R is an alkyl group. Here, an exemplary aminoxyalkyl, depicted as “H₂N—O—R” is added during treatment of the DNA substrate with the DNA glycosylase. Following enzyme-mediated cleavage of the N-glycosidic bond to release the modified nucleobase, the aminoxyalkyl reacts with the abasic site (I) to form a stable adduct (III) that prevents the enzyme from further interacting with the DNA substrate and, e.g., cleaving the phosphodiester backbone.

### Second Complementary Copy Strand

The methods described herein include the step of performing a second primer extension reaction to generate a second complementary copy of the parental DNA template (i.e., a second daughter strand). This step is performed following the enzymatic excision of the modified nucleobases. The second complementary copy of the DNA template thus retains at least a portion of the epigenetic information encoded in the original DNA target fragment.

Following glycosylase treatment, the asymmetrically altered DNA fragments are denatured using any suitable art-recognized method, including acid-base denaturation (using, e.g., acetic acid, HCL, or nitric acid), basic denaturation (using, e.g., NaOH), solvent-based denaturation (using, e.g., DMSO, formamide, guanidine, sodium salicylate, propylene glycol, or urea), or physical denaturation (using, e.g., heat, beads, sonication, or radiation). The resulting single stranded DNA template strands are then purified from the first complementary target strands. Purification of the population of converted template strands is facilitated by the solid-phase synthesis methods described herein, wherein one of the two populations of parent and daughter strands is selectively immobilized on a solid support.

The second primer extension reaction is directed by an extension oligonucleotide hybridized to the DNA target template using a second DNA polymerase to produce a second double stranded DNA fragment that includes a second complementary copy strand hybridized to the parental template strand. The second primer extension reaction may be carried out on a solid support, as described herein, in which either the parent template strand or the second daughter strand is selectively immobilized on the support.

The second DNA polymerase is selected for its ability to synthesize the second complementary copy past the positions of the abasic sites in the converted parental template. DNA polymerases exhibiting this property are known in the art and referred to, e.g., as “bypass”, or “translesion”, polymerases.

In some instances, the second DNA polymerase may be selected based on an activity of preferentially incorporating a specific nucleotide opposite abasic sites in a template. It is an object of the present invention to generate second the complementary copy strands such that the nucleobase incorporated opposite abasic sites in the template do not form Watson and Crick base pairs with the modified nucleobase previously excised from the template. For example, in some instances, the modified base of interest is 5-mC. In this case, a second DNA polymerase is selected based on a preference for incorporating any nucleotide but dGTP (i.e., “Not G”) opposite the positions in which 5-mC has been converted to an abasic site, e.g., the polymerase may preferentially incorporate dATP, dTTP, or dCTP at these sites.

It is known in the art that abasic sites represent the most frequent DNA lesion in the genome and have high mutagenic potential, leading to mutations commonly found in human cancers. Although these lesions are devoid of genetic information, it has been observed that adenine is the most efficiently inserted nucleobase during bypass of abasic sites by DNA polymerases, a phenomenon termed “A-rule”. The strong preference of DNA polymerase for adenine (i.e., dATP) incorporation has been observed for DNA polymerases from family A (including human DNA polymerases γ and θ) and B (including human DNA polymerases α, ε, and δ) (see, e.g., Obeid, et. al. 2010. EMBO J. 29(10): 1738-1747). In a preferred embodiment of the present invention, the second DNA polymerase will have a preference for incorporating A opposite abasic sites in the template, particularly when the modified nucleobase of interest is a derivative of C (e.g., 5-mC).

A non-limiting list of exemplary second DNA polymerases is set forth in Table 3.

TABLE 3
Exemplary Abasic Bypass DNA Polymerases

| DNA polymerase | Species | Family |
|---|---|---|
| gp90 (exo-) | PaP1 phage | A |
| Pol alpha | H. sapiens | A |
| Klenow fragment (exo-) | E. coli | B |
| Vent polymerase (exo-) | T. litoralis | B |
| Klentaq | Chimera | B |
| RB69 pol | RB69 bacteriophage | B |
| gp43 | T4 bacteriophage | B |
| Pol delta | H. sapiens | B |
| Dpo4 | S. solfataricus | Y |
| Dpo4 variants with amino acid | S. solfataricus | Y |
| substitutions | | |
| dinB (Pol IV) | E. coli | Y |
| REV1 | H. sapiens | Y |
| umuC and umuD (Pol V) | E. coli | Y |

In some instances, the second DNA polymerase may include a mixture of more than one DNA polymerase. For example, the mixture may include a DNA polymerase that is capable of incorporating a nucleotide opposite an abasic site, but is incapable of extending the daughter strand further, and another DNA polymerase that does have the capability to extend the daughter strand past the abasic site in the parent strand. In another instance, the mixture may include a DNA polymerase with exonuclease activity. The combination of a bypass polymerase (e.g., DPO4 or a variant thereof) and a polymerase with exonuclease activity (e.g., DPO1), may provide several advantages. For example, the exonuclease may provide error-correcting activity and the combination result in a more efficient and accurate incorporation of the desired nucleotide through, e.g., minimizing polymerase stalls and errors.

In some instances, the substrate preference of a bypass DNA polymerase at abasic sites may be optimized, or directed, by further methods of the present invention. For example, the DNA polymerase may be an engineered variant with mutations that increases its bypass activity or preference for incorporating a specific nucleotide opposite abasic sites.

In one embodiment, the engineered variant is a variant of DPO4 DNA polymerase (SEQ ID NO:1). DPO4 is a DNA polymerase naturally expressed by the archaea, Sulfolobus solfataricus, a Y-family DNA polymerase, which generally function in the replication of damaged DNA by a process known as translesion synthesis (TLS). Advantages of DPO4 include a monomeric structure, open architecture, lack of an exonuclease domain, and ability to bypass abasic sites. The crystal structure of DPO4 is available to guide protein engineering, see, e.g., Ling et al. (2001) “Crystal Structure of a Y-Family DNA Polymerase in Action: A Mechanism for Error-Prone and Lesion-Bypass Replication” Cell 107:91-102. As described in the art, the inventors have engineered thousands of variants of DPO4 that are optimized for, e.g., the ability to utilize unconventional nucleotide analogs as substrates. A non-limiting list of DPO4 variants and screening methodologies that may be used according to the present invention are disclosed in Applicants' issued U.S. Pat. Nos. 11,299,725, 11,530,392, and 11,708,566, the contents of which are herein incorporated by reference in their entireties.

The inventors have previously identified a region of DPO4 polymerase, corresponding to amino acids 76-86, that has been a key target for modifying and optimizing the substrate specificity of the polymerase. Therefore, a number of variants with mutations in this region, in an otherwise wildtype background, were screened for abasic bypass activity with dATP incorporation. From the screen, one particular DPO4 polymerase variant was identified that demonstrates robust abasic bypass activity, and is referred to herein as “C9110”. This variant includes the following mutations, relative to the wildtype polymerase: M76W_K78E_E79P_Q82W_Q83G_S86E and deletion of amino acids 341-352 (SEQ ID NO:3).

### Nucleotide Analogs

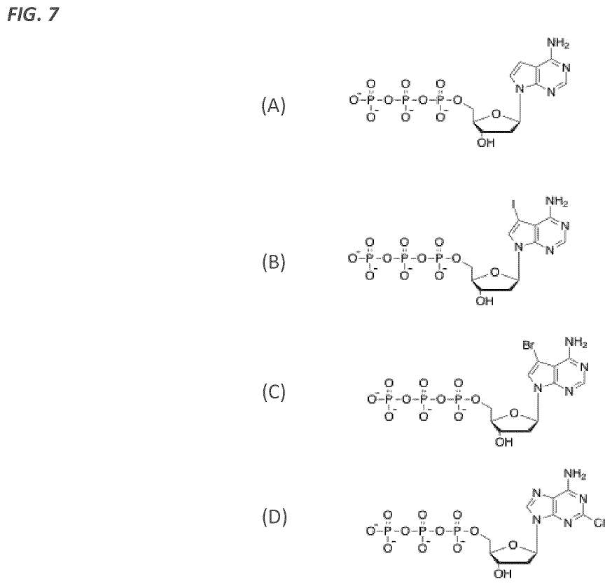

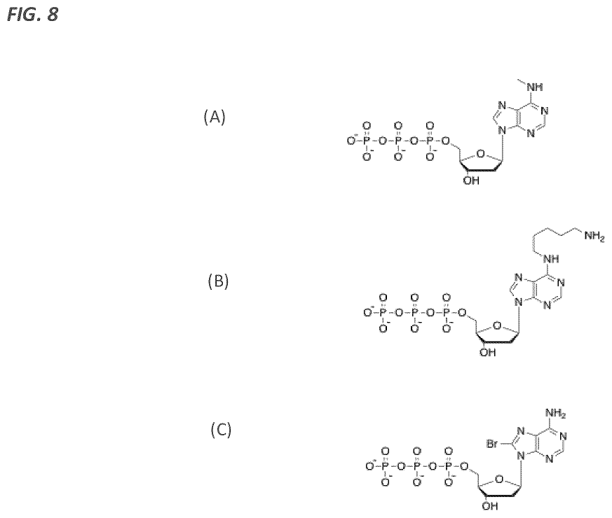

In certain instances, the substrate preference of a bypass DNA polymerase may be modified, or directed, by utilizing alternative nucleotides (i.e., nucleotide analogs) in the second primer extension reaction. For example, when the modified nucleobase of interest is 5-mC, the primer extension reaction may include an analog of dATP, certain examples of which are shown in FIG. 7. For example, the dATP may be, e.g., one or more of DAP (diaminopurine), 7-position substituents such as alkynyl C8, C10, phenyl, or on to 7-deaza dATP, analog (A). Other exemplary dATP analogs include 7-deaza with an iodo, analog (B), or a bromo group bound to the C-7 atom analog (C), or with a chloro group bound to the C-2 atom, analog (D). In other examples, as shown in FIG. 8, dATP may be modified by 6-position substituents, such as N6-methyl dATP, analog (A), N6 aminohex, analog (B), or an 8-Bromo group, analog (C). In one embodiment, N6-methyl dATP is utilized in the second primer extension reaction.

### Designed Nucleotide Analogs

In further aspects, the methods of the present invention may include a nucleotide analog in which the nucleobase is designed to introduce specific structural and/or chemical features that promote incorporation by a bypass DNA polymerase. Exemplary nucleobase features include an overall geometry that is spatially compatible with the empty “pocket” left by excision of a nucleobase. For example, a nucleotide analog that has the size and geometry of two bases (e.g., a base pair) may be advantageous. Other beneficial features may include an overall increase in hydrophobicity or the introduction of a moiety known to enhance incorporation by a bypass polymerase, such as spermine. In certain embodiments, designed nucleotide analogs may include more than one such feature, for example, they may include both a polymerase enhancing feature and a “bulky” hydrophobic feature.

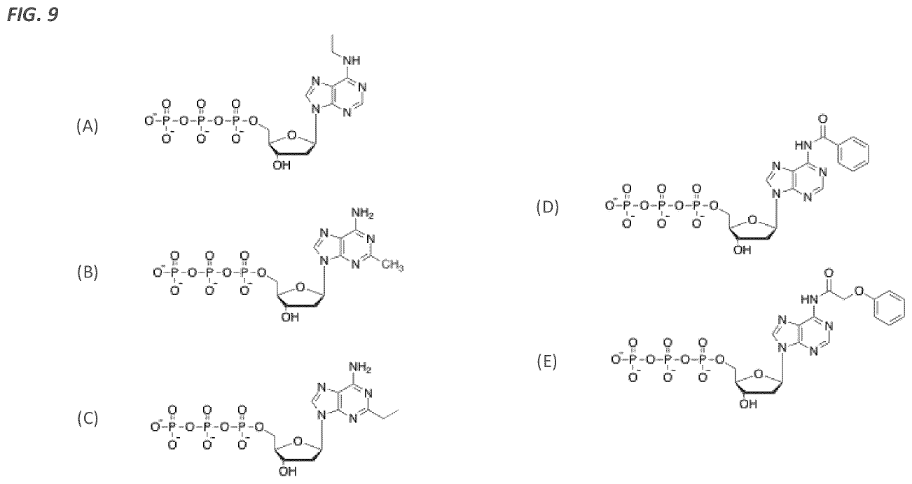

Certain exemplary designed nucleotide analogs include, but are not limited, to the following depicted in FIG. 9: alkyl analogs, N6-Ethyl-2′-dATP, analog (A), 2-Methyl-2′-dATP, analog (B), 2-Ethyl-2′-dATP, analog (C), and protected analogs, N6-Benzoyl-2′dATP, analogs (D), and N6-Phenxoyacetal-2′dATP, analog (E).

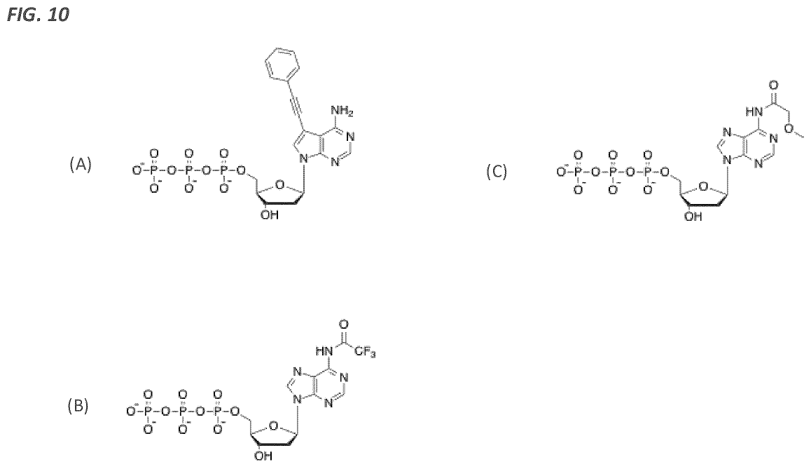

Other exemplary nucleotide analogs include, but are not limited to, the following that are depicted in FIG. 10: 7-Ethynylphenyl-7-deaza-2′-ATP, analog (A), N6-Trifluoroacetamdio-2′-dATP, analog (B), and N6-Ethoxyacetyl-2′dATP, analog (C).

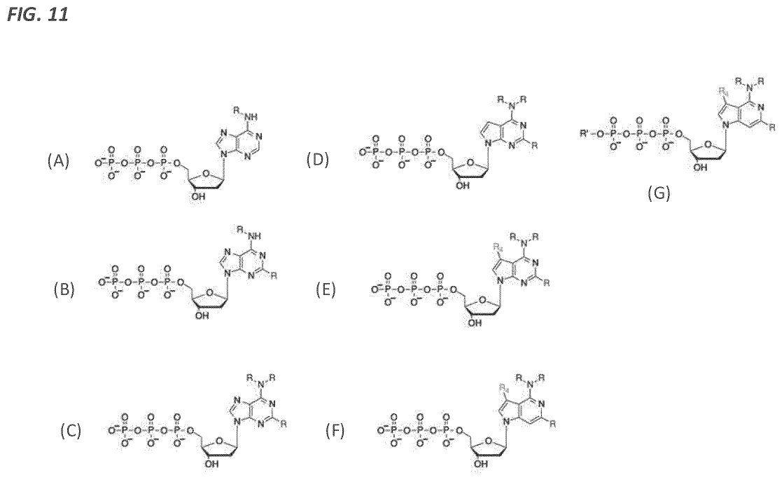

Design of nucleotide, e.g., dATP, analogs suitable for the practice of the present invention may be guided by the generic structures set forth in FIG. 11, which includes the following: N6-(alkyl or acyl)-2′-dATP, compound (A), N6-(alky or acyl)-2-alkyl-2′dATP, compound (B), N6,N6-(alkyl or acyl)-2-alkyl-2′-dATP, compound (C), N6,N6-(alkyl or acyl)-2-alkyl-7-deaza-2′-dATP, compound (D), N6,N6-(alkyl or acyl)-2-alkyl-7-alkynyl-7-deaza-2′-dATP, compound (E), N6,N6-(alkyl or acyl)-2-alkyl-7-alkynyl-3, 7-dideaza-2′-dATP, compound (F), and Gamma-O-alkyl-N6,N6-(alkyl or acyl)-2-alkyl-7-alkynyl-3,7-dideaza-2′dATP, compound (G).

In another embodiment, use of a dGTP analog, such as 7-deaza dGTP, which is a less favorable polymerase substrate, may be influential in determining which nucleotides are incorporated opposite abasic sites during the abasic bypass primer extension reaction. In other embodiments, additional components of the primer extension reaction may be optimized to influence the substrate preference of a bypass DNA polymerase, e.g., buffer pH, solvent compositions, relative ratios of dNTPs, and the like. In some instances, the amount of polymerase protein may be limiting in the reaction, thereby minimizing synthesis of undesired primer extension side-products.

### Aminoxyalkyl Nucleobase Mimetics

As discussed herein, and with reference to FIG. 5, certain chemical stabilizing agents react with abasic sites in DNA to form a stable oxime adduct that prevents subsequent degradation of the phosphodiester backbone. As used herein, the term “oxime” refers to an organic compound belonging to the imines, with the general formula, RR′C═N—OH, where R is an organic side chain and R′ may be hydrogen, forming an aldoxime, or another organic group, forming a ketoxime. O-substituted oximes form a closely related family of compounds. One particularly useful class of stabilizing agents used to form oxime adducts are those with the generalized aminoxyalkyl structure, H₂N—O—R, as disclosed herein. Advantageously, the inventors have discovered that certain oximes have the further capability to biologically mimic the Watson-Crick base-pairing activity of natural nucleobases. Thus, they not only stabilize abasic sites, but also direct incorporation of specific nucleotides at opposing sites during daughter strand synthesis. Such aminoxyalkyl-based stabilizing reagents and their corresponding oxime adduct products may be referred to in certain embodiments herein alternatively as, “nucleobase mimetics”, “aminoxyalkyl nucleobase mimetics”, or “nucleobase oxime mimetics.”

In one embodiment, the uracil mimetic, 1-[2-(amino)ethyl]-uracil, is used to stabilize abasic sites, as the aminoxyalkyl constituent of the mimetic compound reacts with the abasic site to form a stable oxime adduct. Advantageously, the heterocycle constituent of the compound is able to from Watson-Crick base pairs with adenine and will thus direct incorporation of dATP during daughter strand synthesis.

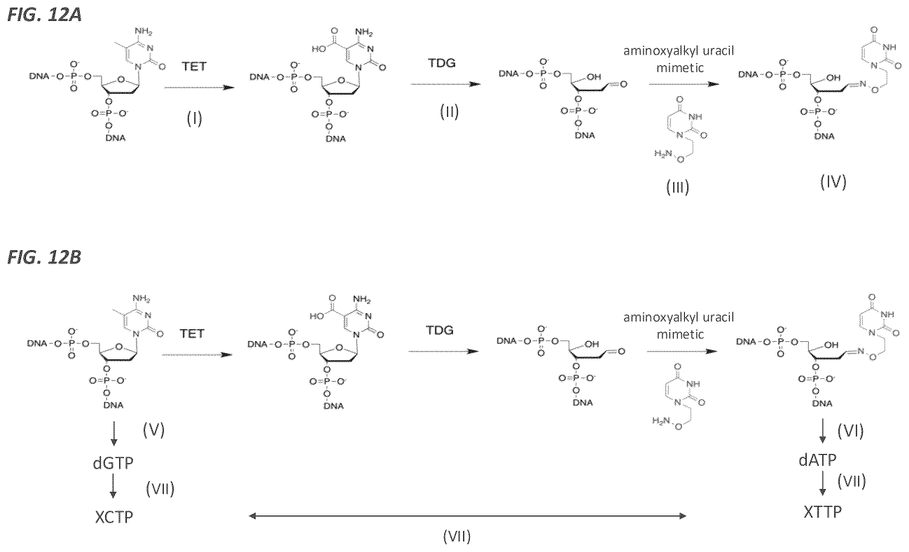

FIG. 12A illustrates one example of the conversion of 5-mC to a uracil oxime mimetic. Here, a DNA target molecule including a 5-mC residue is treated with TET (I) to convert 5-mC to 5-caC, and TDG (II) to excise the 5-caC nucleobase and generate an abasic site, as previously described. In this example, the DNA target is also treated with a aminoxyalkyl uracil mimetic (III), which chemically reacts with the abasic site to form a stable oxime mimetic adduct (IV). In this embodiment, aminoxyalkyl uracil mimetic (III) is 1-[2-(aminooxy)ethyl]-uracil, available from Enamine, Ltd, Kyiv, Ukraine. Advantageously, the inventors have found that both the enzymatic conversion and excision of 5-mC with TET and TDG as well as the chemical conversion of the abasic nucleotide to the stable oxime adduct can be performed in a single reaction, i.e., a “one-pot” reaction. This one-pot reaction is also referred to herein as a “chemo-enzymatic nucleobase conversion reaction”. Importantly, the oxime mimetic adduct (IV) is capable of base-pairing with adenine and thus is read as uracil during daughter strand synthesis.

FIG. 12B illustrates how chemo-enzymatic conversion of 5-mC to the uracil oxime mimetic can be used in the detection of 5-mC in a DNA target fragment. Here, as discussed with reference to FIG. 12A, a parental DNA template is subjected to steps (I) through (IV) to chemo-enzymatically convert 5-mC to the uracil oxime mimetic. Prior to this conversion, a first daughter strand copy of the template is synthesized (V), as discussed with reference to FIG. 1B. This reaction is carried out with native nucleotides, such that native G is incorporated into the daughter strand opposite positions of 5-mC in the parental template. Following chemo-enzymatic conversion of the parental template, the second primer extension reaction generates the second daughter strand copy (VI). This reaction may also be carried out with native nucleotides, such that native A is incorporated at positions opposite the uracil oxime mimetic. For sequence comparison analysis, both the first and second daughter strand copies serve as templates for the Sequencing by Expansion (SBX®) protocol (VII), as described further herein. The resulting sequencing reads of the first daughter strand copy will indicate “C” at each of the positions of 5-mC in the original parental template, while the sequencing reads of the second daughter strand copy will indicate “T” at each of the positions of 5-mC in the parental template. Thus “C->T” substitutions in the sequence of the Xpandomer copy of second daughter strand reveal the positions of 5-mC in the target fragment.

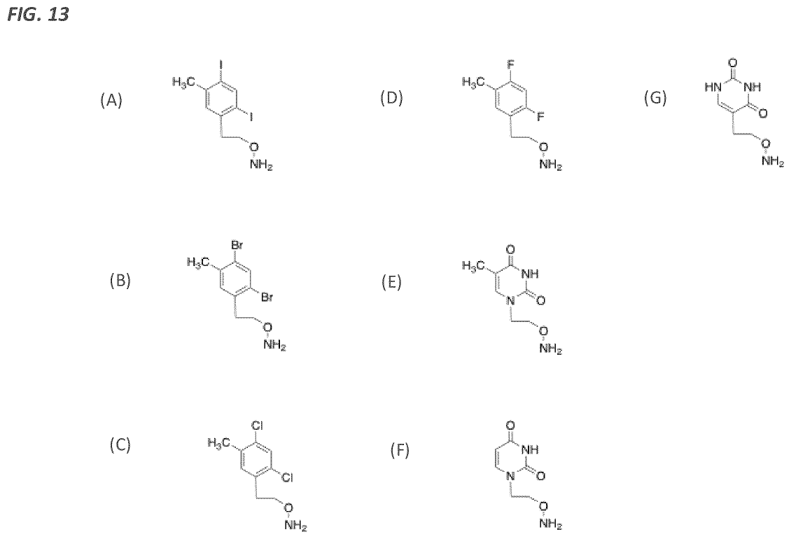

Other exemplary aminoxyalkyl nucleobase mimetics suitable for the methods of the present invention include 1-[3-(aminoxy)propyl]-uracil, 1-[4-(aminoxy)butyl]-uracil, 1-[5-(aminoxy)pentyl]-uracil, commercially available from, e.g., Enamine Ltd. In other embodiments, the present invention contemplates new aminoxyalkyl nucleobase mimetics in which certain chemical features are optimized for particular applications. For example, mimetics may include heterocycles other than uracil, such as thymine, cytosine, guanine, or adenine. In other embodiments, the mimetics may include alternative atomic distances between the oxime and the heterocycle, e.g., from two carbons to three, four, or five carbons. Certain exemplary aminoxyalkyl nucleobase mimetics are set forth in FIG. 13 and include the following: 1-[2-(aminoxy)ethyl]-2,4-diiodo-5-methyl benzene, compound (A); 1-[2-(aminoxy)ethyl]-2,4-dibromo-5-methyl benzene, compound (B); 1-[2-(aminoxy)ethyl]-2,4-dichloro-5-methyl benzene, compound (C); 1-[2-(aminoxy)ethyl]-2,4-difluoro-5-methyl benzene, compound (D); 1-[2-(aminoxy)ethyl]-thymine, compound (E); and further prophetic pseudo uridine analogs, compounds (F) and (G).

According to the present invention, in cases where a nucleotide has been incorporated in the second complementary strand at a position opposite an abasic site in the DNA target strand, it is preferentially incapable of forming a Watson-Crick base pair with the original excised modified nucleobase under the primer extension conditions described herein. For example, if the modified nucleobase of interest is a derivative of cytosine, the nucleotide incorporated opposite the excised base will not be dGTP, but will rather be dATP, dCTP, or dTTP, or derivatives thereof; if the modified nucleobase of interest is a derivative of guanine, the nucleotide incorporated opposite the excised base will not be dCTP, but will rather be dATP, dGTP, or dTTP, or derivatives thereof; if the modified nucleobase of interest is a derivative of adenine, the nucleotide incorporated opposite the excised base not be dTTP, but will rather be dATP, dCTP, or dGTP or derivatives thereof; if the modified nucleobase of interest is a derivative of thymine, the nucleotide incorporated opposite the excised base will not be dATP, but will rather be dCTP, dGTP, or dTTP or derivatives thereof. In a preferred embodiment, as discussed herein, native dATP, or derivative thereof, is the nucleotide incorporated opposite abasic sites resulting from the excision (i.e., conversion) of modified cytosine (e.g., 5-mC) in the original DNA target fragment.

In some instances, the yield of the desired incorporated nucleotides is at least about 50%, 55%, 60%, 65%, 70%, 75%, 80%, 85%, 90%, 95%, or nearly 100% of the total number of incorporation events for each second complementary copy strand produced. For example, the yield of the desired incorporated nucleotide may be at least 50%, 55%, 60%, 65%, 70%, 75%, 80%, 85%, 90%, 95%, or nearly 100% of the total events in each second primer extension reaction. In one example, the yield of the desired incorporated products may be at least 80%. In one example, the yield of the desired incorporated nucleotides may be at least 85%. In another example, the yield of the desired incorporated nucleotides may be at least 90%. In another example, the yield of the desired incorporated nucleotide may be at least 95%. In another example, the yield of the desired incorporated nucleotide may be nearly 100%.

In certain instances, the second DNA polymerase may “skip” over an abasic site during the second primer extension reaction and create a deletion in the second complementary copy opposite the position of an abasic site. In yet other instances, the second DNA polymerase may incorporate more than one nucleotide at a position opposite the abasic site in the target DNA polymerase, thus creating an insertion in the second complementary copy. In either case, the sequence of the second daughter strand includes differences from the sequence of the first daughter strand that inform the positions of modified nucleobases in the target fragment.

In some instances, once the first and second complementary copy strands of the DNA target fragment are produced as described above, they can be assessed through a number of established and emerging nucleic acid sequencing techniques, including, but not limited to, deep sequencing, next generation sequencing, and nanopore sequencing.

### Chemo-Enzymatic Nucleobase Conversion Reaction Mixtures

In certain aspects, a chemo-enzymatic nucleobase conversion reaction mixture according to the present invention may include at least one DNA glycosylase enzyme, a chemical stabilizing agent, and a suitable buffer.

Each DNA glycosylase may have specificity for one or more different kinds of modified nucleobases or one or more types of nucleobase modification. In some embodiments, the DNA glycosylase enzyme includes one of the glycosylase enzymes as set forth in Table 1. In other embodiments, the chemo-enzymatic nucleobase conversion reaction mixture may include an additional enzyme that chemically converts a modified nucleobase of interest, while not excising the nucleobase from a DNA fragment, e.g., a TET enzyme. In some embodiments, the amount of DNA glycosylase enzyme in the nucleobase conversion reaction mixture will be an amount sufficient to completely excise the majority of the modified nucleobases of interest from a DNA target fragment. For example, the amount of DNA glycosylase enzyme may be around 0.1 μg purified enzyme protein/pmol DNA template, around 0.15 μg purified enzyme protein/pmol DNA template, around 0.2 μg purified enzyme protein/pmol DNA template, around 0.3 μg purified enzyme protein/pmol DNA template, around 0.5 μg purified enzyme protein/pmol DNA template, around 0.7 μg purified enzyme protein/pmol DNA template, around 1.0 μg purified enzyme protein/pmol DNA template, around 1.5 μg purified enzyme protein/pmol DNA template, around 2 μg purified enzyme protein/pmol DNA template, or over 2 μg purified enzyme protein/pmol DNA template.

In some embodiments, the chemical stabilizing agent may be selected from the group consisting of 1-[2-(amino)ethyl]-uracil, 1-[3-(aminoxy)propyl]-uracil, 1-[4-(aminoxy)butyl]-uracil, 1-[5-(aminoxy)pentyl]-uracil, 1-[2-(aminoxy)ethyl]-2,4-diiodo-5-methyl benzene, 1-[2-(aminoxy)ethyl]-2,4-dibromo-5-methyl benzene, 1-[2-(aminoxy)ethyl]-2,4-dichloro-5-methyl benzene, 1-[2-(aminoxy)ethyl]-2,4-difluoro-5-methyl benzene, and 1-[2-(aminoxy)ethyl]-thymine. In some embodiments, the chemical stabilizing agent may be present in the nucleobase conversion reactions mixture at a final molarity of around 1 mM, around 5 mM, around 10 mM, around 15 mM, around 20 mM, around 25 mM, around 30 mM, up to 50 mM, up to 75 mM, up to 100 mM, or over 100 mM.

In some embodiments, the suitable buffer may be selected from the group consisting of MES, Tris-HCl, HEPES, and the like. In further embodiments, the suitable buffer may include additional excipients, such as a salt (e.g., NaCl or NaOAc), DTT, MgCl₂, DTT, PEG, and the like. In other embodiments, the nucleobase conversion reaction may include co-factors suitable for a particular DNA glycosylase or other conversion enzyme, for example one or more of ammonium iron(II) sulfate, alpha ketoglutarate, and sodium ascorbic acid. In some embodiments, the final pH of the nucleobase conversion reaction mixture may be around pH 4, around pH 5, around pH 6, around pH 7, or above pH 7. Of course, one of skill in the art will appreciate that the final pH will depend upon the particular stabilizing agent, DNA glycosylase, and other enzymes present in the reaction mixture.

In certain embodiments, the chemo-enzymatic nucleobase conversion reaction mixture may be a liquid, a frozen liquid, a dried liquid, a lyophilized liquid, or a partially lyophilized liquid.

### Kits

In another aspect, kits comprising reagents for performing the methods as described herein are provided. In certain embodiments, the kits may include a chemo-enzymatic nucleobase conversion reaction mixture, as described herein. Various other enzymes may be included in the kit. For example, the kit may include one or more of a high fidelity DNA polymerase, an abasic bypass DNA polymerase, and a DNA polymerase with exonuclease activity. The kit may also include a DNA ligase for library preparation, e.g., the ligation of adapters to the DNA target fragments to create a library of adapter-ligated DNA target fragments.

In some instances, the kit may include one or more buffers and/or reaction components for performing the first primer extension reaction, nucleobase excision reaction, abasic stabilization reaction, and second primer extension reaction steps of the method. For example, the kits may include one or more of a DNA polymerase buffer, a DNA glycosylase buffer, a DNA ligase buffer, or any combination thereof. The kit may also include other reagents such as salts, cations, or detergents.

In some instances, the kits includes reagents and instructions for fragmentation of the DNA sample and ligation of adapters. For example, the kit may include one or more enzymes for fragmenting the DNA and ligation of adapters.

In some instances, the kit may further include control DNA oligonucleotides containing one or more of the modified nucleobases of interest. The control oligonucleotides may be provided in a known concentration and having a known amount of modified nucleobase per DNA molecule or concentration. In some instances, the control DNA oligonucleotide may be in a specific size range. For example, the control DNA oligonucleotides may be in the range of 25-100 bp, 25-150 bp, 50-200 bp, 50-300 bp, 25-500 bp and so on. In some instances, the control DNA oligonucleotides may be in the same approximate size range as the DNA molecules to be analyzed using the kit.

In some instances, the kit may further include instructions. The instructions may specific how to perform one or more of the DNA isolation step, the DNA fragmentation step, the adapter ligation step, the first primer extension reaction step, the glycosylase treatment step, the abasic site stabilization step, and the second primer extension reaction step. Instructions describing how to use control DNA oligonucleotides may also be included in the kit.

### Sequencing by Expansion

One nucleic acid sequencing methodology of the present invention is “Sequencing by Expansion” (SBX®), developed by Stratos Genomics (see, e.g., Kokoris et al., U.S. Pat. No. 7,939,259, “High Throughput Nucleic Acid Sequencing by Expansion”, which is herein incorporated by reference in its entirety). SBX is based on the polymerization of highly modified, non-natural nucleotide analogs, referred to as “XNTPs”. In general terms, SBX uses biochemical polymerization to transcribe the sequence of a DNA template (e.g., the first and second complementary copies of the DNA target fragments, as described herein) onto a measurable polymer called an “Xpandomer”. The transcribed sequence is encoded along the Xpandomer backbone in high signal-to-noise reporters that are separated by ~10 nm and are designed for high-signal-to-noise, well-differentiated responses. These differences provide significant performance enhancements in sequence read efficiency and accuracy of Xpandomers relative to natural DNA.

XNTPs are expandable, 5′ triphosphate modified non-natural nucleotide analogs compatible with template dependent enzymatic polymerization. The XNTP has two distinct functional regions; namely, a selectively cleavable phosphoramidate bond, linking the 5′ α-phosphate to the nucleobase, and a symmetrically synthesized reporter tether (SSRT) that is attached within the nucleoside triphosphoramidate at positions that allow for controlled expansion by cleavage of the phosphoramidate bond. The SSRT includes linkers separated by the selectively cleavable phosphoramidate bond. Each linker attaches to one end of a reporter code. XNTP substrates incorporated into daughter strand products of template-dependent polymerization are in the “constrained” configuration. The constrained configuration of polymerized XNTPs is the precursor to the expanded configuration, as found in Xpandomer products.

The transition from the constrained configuration to an expanded configuration results from cleavage of the selectively cleavable phosphoramidate bonds within the primary backbone of the daughter strand. In this embodiment, the SSRTs include one or more reporters or reporter codes, specific for the nucleobase to which they are linked, thereby encoding the sequence information of the template. In this manner, the SSRTs provide a means to expand the length of the Xpandomer and lower the linear density of the sequence information of the parent strand.

The SSRT (i.e., “tether”) of the XNTP includes several distinct functional elements, or features, such as polymerase enhancement regions, reporter codes, and translation control element (TCEs). Each of these features performs a unique function during translocation of the Xpandomer through a nanopore to produce a series of unique and reproducible electronic signal. The SSRT is designed for controlling the rate of Xpandomer translocation by the TCE through a combination of sterics and/or electrorepulsion, different reporter codes are sized to block ion flow through a nanopore at different measurable levels.

Specific SSRT polymeric sequences can be efficiently synthesized using phosphoramidite chemistry typically used for oligonucleotide synthesis. Reporter codes and other features can be designed by selecting a sequence of specific phosphoramidites from commercially available and/or proprietary libraries. Such libraries include, but are not limited to, polyethylene glycol with lengths of 1 to 12 or more ethylene glycol units and aliphatic polymers with lengths of 1 to 12 or more carbon units. In certain embodiments, the SSRTs include features referred to as “polymerase enhancement regions” at the ends of the SSRTs proximal to the nucleotide triphosphoramidate diester. Polymerase enhancement regions may include positively charged polyamine spacers (e.g., primary, secondary, tertiary, or quaternary amines) or triamine spacers (three secondary amines each separated by three carbons) that facilitate incorporation of XNTP structures by a nucleic acid polymerase. In certain embodiments, the polymerase enhancement region includes two repeat units spermine

As used throughout the present disclosure, the terms “linker A” and “linker B” refer to the regions of the SSRT that each include a polymerase enhancing region and one or more translocation deceleration features or regions, and, in certain embodiments, a spacer region that includes a polymer of, e.g., PEG6, which can be customized to modulate the length of the SSRT traversed in a nanopore.

In certain embodiments, an XNTP may be a compound having the following generalized structure:

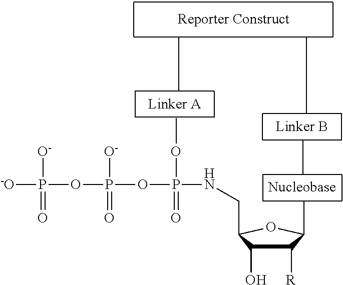

In one embodiment, R may be H, for example, when the compounds are used to sequence a DNA template.

In certain embodiments, nucleobase is adenine, cytosine, guanine, thymine, uracil or a nucleobase analog. As one of skill in the art will appreciate, adenine, cytosine, guanine, thymine, and uracil are naturally occurring nucleobases. As used herein, the term “nucleobase analog” refers to non-naturally occurring nucleobases that are capable of forming Watson and Crick base pair with a complementary nucleobase on an adjacent single-stranded nucleic acid template.

To obtain sequence information, an Xpandomer is translocated through a nanopore, from the cis reservoir to the trans reservoir. As the Xpandomer translocates, a reporter enters the stem until its translocation control element stops at the stem entrance. The reporter is held in the stem until the TCE is enabled to pass into and through the stem, whereupon translocation proceeds to the next reporter. Upon passage through the nanopore, each of the reporter codes of the linearized Xpandomer generates a distinct and reproducible electronic signal, specific for the nucleobase to which it is linked.

In certain embodiments, Xpandomers produced by the SBX chemistry may be analyzed using a nanopore-based sequencing chip. A nanopore-based sequencing chip can incorporate a large number of sensor cells configured as an array. For example, the chip may include an array of one million cells configured in 1000 rows by 1000 columns of cells. Each cell in the array may include a control circuit integrated on a silicon substrate. Such nanopore-based sequencing chips, devices, and systems are described, e.g., in Applicant's published patent application no. WO2021/219795, which is herein incorporated by reference in its entirety.

Proprietary in-house bioinformatics pipelines are typically used to process sequencing reads. The methods disclosed herein leverage UMIs to enable pairing of first and second complementary copy reads. Read pairs may be quality filtered and trimmed of adapter and primer sequences. UMI sequences may be clustered together, defining UMI-families (all reads originating from a single DNA template).

### Diagnostic and Prognostic Methods

In particular embodiments, the methods can be directed to diagnosing an individual with a condition that is characterized by a methylation level and/or pattern of methylation at particular loci in a test sample that are distinct from the methylation level and/or pattern of methylation for the same loci in a sample that is considered normal or for which the condition is considered to be absent. The methods can also be used for predicting the susceptibility of an individual to a condition that is characterized by a level and/or pattern of methylated loci that is distinct from the level and/or pattern of methylated loci exhibited in the absence of the condition.

With particular regards to cancer, changes in DNA methylation have been recognize as one of the most common molecular alterations in human neoplasia. Hypermethylation of CpG islands located in promoter regions of tumor suppressor genes is a well-established and common mechanism for gene inactivation in cancer (Esteller, Oncogene 21(35): 5427-40 (2002)). In contrast, a global hypomethylation of genomic DNA is observed in tumor cells; and a correlation between hypomethylation and increased gene expression has been reported for many oncogenes (Feinberg, Nature 301(5895): 89-92 (1983), Hanada, et al., Blood 82(6): 1820-8 (1993)). Cancer diagnosis or prognosis can be made in a method set forth herein based on the methylation state of particular sequence regions of a gene including, but not limited to, the coding sequence, the 5′-regulatory regions, or other regulatory regions that influence transcription efficiency.

A reference genomic DNA (for example, gDNA considered “normal”) and a test genomic DNA that are to be compared in a diagnostic or prognostic method, can be obtained from different individuals, from different tissues, and/or from different cell types. In particular embodiments, the genomic DNA samples to be compared can be from the same individual but from different tissues or different cell types, or from tissues or cell types that are differentially affected by a disease or condition. Similarly, the genomic DNA samples to be compared can be from the same tissue or the same cell type, wherein the cells or tissues are differentially affected by a disease or condition.

### EXAMPLES

### Example 1

### A One-Pot Chemo-Enzymatic Conversion Reaction

This Example demonstrates glycosylase-mediated excision of 5-mC from a double stranded DNA target fragment and chemical conversion of the resulting abasic sites into stable oxime adducts, utilizing a aminoxyalkyl uracil mimetic. Advantageously, the enzymatic and chemical conversion reactions were carried out simultaneously in a single reaction vessel (i.e., a “one-pot” reaction).

For this experiment, a single stranded DNA target fragment (80mer) was designed to include three spaced 5-mC residues. The 5′ end of the target strand was covalently modified with biotin to facilitate physical manipulation of the strand with streptavidin-coated beads. The target strand was hybridized to a complementary oligonucleotide strand including native nucleotides at a molar ratio of 5:7.5 pmol, to produce a double stranded fragment. A 21mer oligonucleotide primer was designed to hybridize to the 3′ end of the template.

The “one-pot” conversion reaction included the following reagents: the double stranded DNA fragment, 3 μg purified ngTET protein, 8 μg purified TDG protein, 50 mM MES buffer, pH 6, 50 mM NaCl, 1 mM alpha ketoglutarate (TET cofactor), 2 mM sodium ascorbic acid (TET cofactor), 1 mM DTT, 20% PEG, 0.1 mM ammonium iron(II) sulfate (Mohrs' salt, TET cofactor), and either 10 mM or 26 mM of the aminoxyalkyl uracil mimetic, 1-[2-(aminooxy)ethyl]-4-hydroxy-1,2-dihydropyrimidin-2-one (C₆H₉N₃O₃), commercially available from Enamine, Ltd., Kyiv, Ukraine. The final reaction (50 μL) was incubated at 28° C. for 3 hr. Controls included similar one-pot reactions, but excluding the uracil mimetic and additionally excluding the TET and TDG enzymes.

To enable detection of chemo-enzymatic conversion of the target strand, reaction products were subjected to mild basic conditions (100 mM NaOH for 20′) to selectively cleave the target strand at newly generated abasic sites. Reaction products were analyzed by gel electrophoresis and visualized by cyberstain.

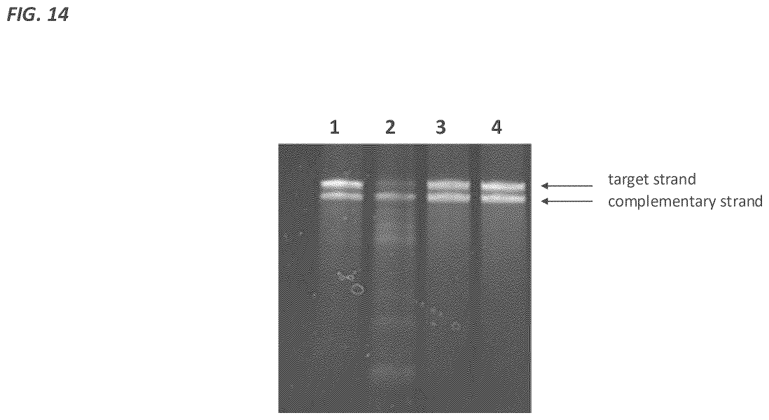

A representative gel is shown in FIG. 14. Lane 1 shows the products of the control reaction, lacking the TET and TDG proteins. The larger band corresponds to the longer target strand and the smaller band corresponds to the shorter complementary strand. As expected, no degradation of the target strand was observed in the absence of DNA glycosylase enzyme. In contrast, lane 2 shows degradation of the target strand in the presence of TET and TDG protein, indicating that the 5-mC residues are being excised to generate unstable abasic sites that are susceptible to base-mediated strand degradation. Significantly, lanes 3 and 4 show that inclusion of the aminoxyalkyl uracil mimetic in the conversion reaction prevents target strand degradation. This observation is consistent with a mechanism by which the mimetic forms stable oxime adducts at the abasic sites created by excision of the nucleobase that are refractory to further degradation.

These results demonstrate the successful chemo-enzymatic conversion of 5-mC residues to stable oxime adducts in a DNA target fragment and provide proof-of-concept support that these discrete reactions can be carried out in a single, one-pot reaction.

### Example 2

### DPO4 Polymerase Exhibits Abasic Bypass Activity

This example demonstrates that DPO4, a class Y DNA polymerase isolated from S. solfataricus, is capable of successfully synthesizing full-length copies of a DNA template that includes several abasic challenges.

For this experiment, a single stranded 80mer template was designed to include three abasic (AP) sites. The 5′ end of the template was covalently modified with a biotin moiety for immobilization of the template on streptavidin-coated beads. A 21mer extension oligonucleotide (EO) was designed to hybridize to the 3′ end of the template. The 5′ end of the EO was covalently modified a SIMA dye for fluorescent detection of primer extension products.

Prior to the primer extension reaction, the template was prepared by incubating 75 pmol of template with 100 pmol EO and 50 μl (10 mg/ml) of beads (Dynabeads™ MyOne™ Streptavidin C1, Thermofisher, Inc.) and incubated at room temperature for 10 minutes.

For a given primer extension reaction, 10 μl of the DNA-bead complex was used to provide the template and EO. The primer extension reaction included the following reagents: 20 mM Tris-HCl, pH 8.8, 10 mM (NH₄)2SO₄, 10 mM KCl, 2 mM MgSO₄, 0.1% Triton X-100, 200 μM dNTPs, 1 mM MnCl, and 2 μg purified DPO4 polymerase. The total reaction volume was 20 μl. Reactions were run for I hour at 37 degrees C. Primer extension products were analyzed by gel electrophoresis following elution of the products from the beads with a buffer containing NaOH.

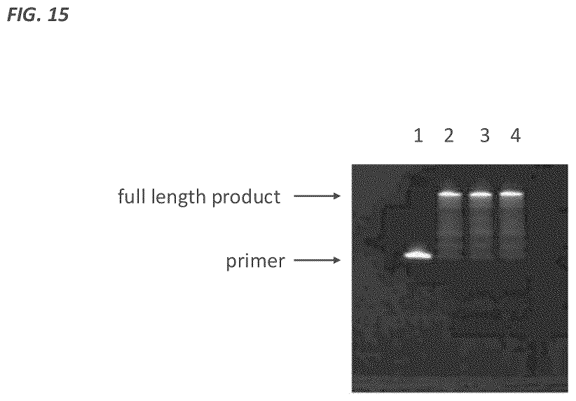

A representative gel is shown in FIG. 15. Lane 1 shows the products of a primer extension reaction lacking the DNA polymerase. As expected, no extension products are observed. Lanes 2-4 show the products of primer extension reactions including no further additives (lane 2) or including 50% 7-deaza dGTP (lane 3) or 100% 7-deaza dGTP (lane 4). As shown, DPO4 polymerase was able to effectively synthesize full length copies of the 80mer template, indicating that it is surprisingly capable of bypassing all three abasic sites in the DNA template.

These results indicate that DPO4 is capable of synthesizing daughter strands past several abasic sites in a parent template and thus validate this enzyme as a potentially suitable polymerase for practice of the methods disclosed herein.

### Example 3

Improved Bypass Activity on a DNA Template with Stabilized Abasic Sites

This example demonstrates that the combination of an engineered DPO4 variant and wildtype DPO1 polymerases is capable of synthesizing a full-length copy of a DNA template that includes three abasic sites stabilized as uracil oxime mimetics. Moreover, this example demonstrates that stabilization of the abasic sites as uracil oxime mimetics directs efficient incorporation of dATP at opposing sites in a newly synthesized daughter strand.

For this experiment, a single stranded DNA template (80mer) was designed to include three abasic (AP) sites spaced relatively evenly along the length of the template. The abasic oligonucleotide was synthesized with conventional phosphoramidite chemistry using the Abasic II phosphoramidite (5-O-Dimethoxytrityl-1-O-tert-butyldimethylsilyl-2-deoxyribose-3-[(2-cyanoethyl)-(N,N-diisopropyl)]-phosphoramidite), available from, e.g., Glen Research, Sterling, VA, according to the manufacturer's recommended protocol. The abasic oligonucleotide was treated with 100 mM aminoxyalkyl at pH 4-5 to generate oxime adducts at the abasic sites and purified by gel electrophoresis. This experiment utilized the aminoxyalkyl uracil mimetic as described in Example 1.

The 5′ end of the template was conjugated with biotin to enable physical manipulation of the strand. A 21mer extension oligonucleotide (EO) was designed to hybridize to the 3′ end of the template. The 5′ end of the EO was covalently modified with a SIMA dye for fluorescent detection of primer extension products.

The following primer extension reactions using the abasic oligonucleotide as a template were conducted: A) extension with KAPA DNA polymerase, B) extension with wildtype DPO4 polymerase, C) extension with DPO4 polymerase variant, C9110, and D) extension with the combination of DPO4 variant polymerase, C9110, and DPO1 polymerase.

Primer extension reaction A included the following reagents: 3 pmol abasic template, 2 pmol extension oligo primer, KAPA HiFi buffer and polymerase, available from Roche Sequencing Solutions. The total reaction volume was 10 μl. Reactions were run for 30 minutes at 55 degrees C., according to the manufacturer's instructions. As a control, an identical primer extension reaction was carried-out using a native template with no abasic sites. Primer extension reaction B included the following reagents: 3 pmol abasic template, 2 pmol extension oligo primer, 20 mM Tris-HCl, pH 8.8, 10 mM (NH₄)2SO₄, 10 mM KCl, 2 mM MgSO₄, 0.1% Triton X-100, 200 μM dNTPs, and 2 μg purified DPO4 polymerase. The total reaction volume was 10 μl. Reactions were run for I hour at 37 degrees C. Primer extension reaction C included the following reagents: 3 pmol abasic template, 2 pmol extension oligo primer, 20 mM Tris-HCl, pH 8.8, 100 mM NaCl, 20 μM dNTPs/1000 μM dATP, 1 μg purified DPO4 polymerase variant C9110, 4 mM MgCl₂, 10% PEG, 10% BHA NMP, 150 mM betaine, 1 mM spermine, 0.15 mM HMP, 1 mM PEM. The total reaction volume was 10 μl. Reactions were run for 14 hours at 55 degrees C. Primer extension reaction D included the following reagents: 3 pmol abasic template, 2 pmol extension oligo primer, 20 mM Tris-HCl, pH 8.8, 100 mM NaCl, 20 μM dNTPs/1000 μM dATP, 1 μg purified DPO4 variant C9110, 25 nM Dpo1, 4 mM MgCl₂, 10% PEG, 10% BHA NMP, 150 mM betaine, 1 mM spermine, 0.15 mM HMP, 1 mM PEM. The total reaction volume was 10 μl. Reactions were run for 14 hours at 55 degrees C. Primer extension products were analyzed by gel electrophoresis and visualized by excitation of the SIMA(HEX) dye linked to the extension oligo.

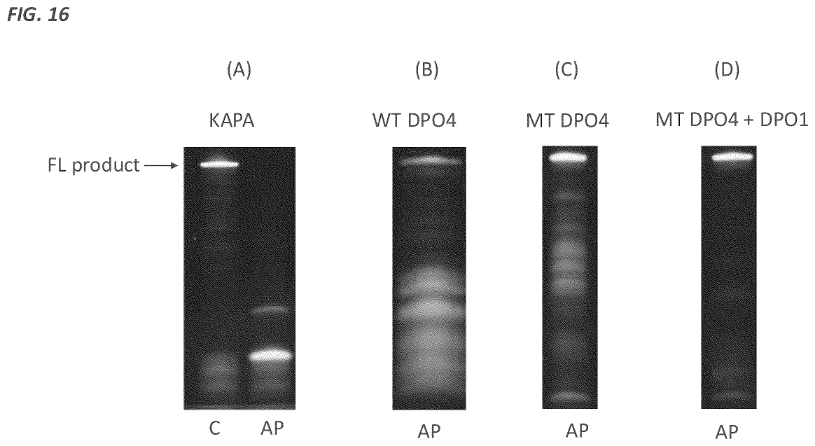

Representative gels are shown in FIG. 16. As shown on gel (A), the KAPA polymerase is able to synthesize a full-length (FL) copy of the native 80mer template (lane “C”); however, this polymerase is unable to extend the extension oligo hybridized to the abasic template (lane “AP”), as the small fluorescent band observed in the gel indicates that the polymerase stalls at the first abasic site in the template. In contrast, as shown on gel (B), wildtype DPO4 polymerase is capable of synthesizing full-length copies of the abasic template, as evidenced by the large band corresponding to the full-length product in the gel. However, the wildtype polymerase also stalls at the abasic sites in the template, as demonstrated by the smear of incomplete extension products in the gel. As shown on gel (C), the DPO4 variant, C9110, demonstrates improved extension activity relative to the wildtype polymerase, with more efficient synthesis of full-length copies of the abasic template. As shown on gel (D), the combination of the DPO4 variant, C9110, and DPO1 polymerase demonstrates the most significant improvement in primer extension activity, as most of the extension products observed by gel are full-length in size. Without being bound by theory, it is speculated that the exonuclease activity of DPO1 may function as a “correction factor,” e.g., by reversing misincorporations made by DPO4 and allowing the polymerase to resume with higher fidelity extension.

These results indicate that the combination of the DPO4 variant, C9110, and DPO1 polymerases is capable of synthesizing full-length daughter strands past several abasic adducts in the parental template with improved efficiency relative to the wildtype DPO4 or DPO4 variant polymerases alone.

To identify the nucleotides incorporated by the DPO4 variant and DPO1 polymerases at sites opposite the abasic sites in reaction D described above, the products of this primer extension reaction were subjected to DNA sequence analysis. The particular DNA sequencing methodology utilized was the nanopore-based Sequencing by Expansion method developed by the inventors, which has been described in more detail above.

To synthesize Xpandomer copies of the primer extension products, an SBX reaction was carried-out that included the following reagents: a 2:1 molar ratio of single stranded DNA template to SBX extension oligonucleotide, 0.07 μg/μL DNA polymerase (DPO4 variant C7326, SEQ ID NO:2) 15 mM AZ-43,43 PEM (i.e., compound 73 as disclosed in Applicant's published PCT Application No. WO2019/135975, which is herein incorporated by reference in its entirety), 100 μM XNTPS (as disclosed in Applicant's published PCT Application No. WO2020/236526, which is herein incorporated by reference in its entirety), 0.2 mM HMP, 0.6 mM MnCl₂, 50 mM Tris HCl, 175 mM NaCl, 200 mM imidazole, 350 mM betaine, 20% PEG, 7% NMP, 3% DMSO. The reaction was run for 2 hours at 37 degrees C. The resulting Xpandomer sample was treated with acid (7.5M DCl) to cleave the phosphoramidate bonds within the XNMP subunits and generate the expanded form of the Xpandomer. The Xpandomers were sequenced using the Roche HTP High Throughput Nanpore Sequencing Platform, as described, e.g., in Applicant's Published PCT Application No. PCT/EP2019/084581, which is herein incorporated by reference in its entirety.

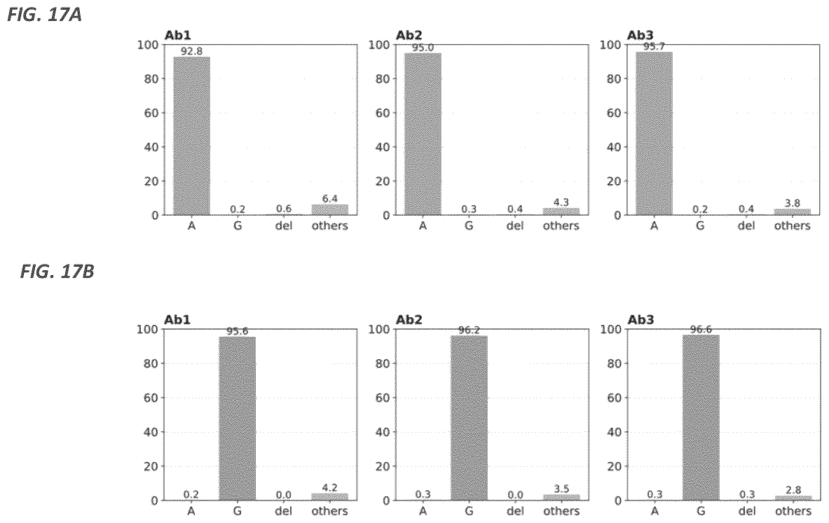

For this experiment, over 10⁶ individual full-length Xpandomer sequences were obtained and analyzed. The results of these analyses are presented in FIGS. 17A and 17B, which are graphs depicting the percentage of the total sequences showing a particular nucleotide incorporation at each of the three abasic sites in the parental DNA template. Significantly, as shown in FIG. 17A (corresponding to primer extension reaction D), dATP was by far the most efficiently incorporated nucleotide opposite each of the abasic sites in the template, with over 90% of the primer extension product sequences showing A at each of these three positions. Furthermore, incorporation of dGTP at any of these positions was observed to be a very rare event. In contrast, as shown in FIG. 17B (corresponding to primer extension reaction A), dGTP was by far the most efficiently incorporated nucleotide opposite each of the 5-mC residues in the native template, as expected.

In sum, these results demonstrate the chemo-enzymatic conversion of 5-mC resides to uracil mimetic adducts in a DNA template and, advantageously, the efficient incorporation of dATP opposite these sites by the combination of an engineered DPO4 variant and DPO1 polymerases. This novel conversion strategy enables identification of G->A substitutions when sequence reads of first and second daughter strand copies of the unconverted and converted DNA template, respectively, are compared and thus offers an improved alternative approach to the identification of epigenetic information in DNA samples.

### SPECIFICALLY INCLUDED EMBODIMENTS

The following embodiments are specifically contemplated as part of the disclosure. This is not intended to be an exhaustive listing of potentially claimed embodiments included within the scope of the disclosure.

Embodiment 1. A method of identifying a modified nucleobase in a plurality of nucleic acids, the method comprising: providing a sample comprising a plurality of DNA templates; generating first complementary copies of the DNA templates, the generating being directed by an oligonucleotide primer using a first DNA polymerase in the presence of native dNTPs, wherein the generating produces a complementary copy of each of the DNA templates such that each complementary copy comprises native dNTPs, and wherein each complementary copy is hybridized to one of the DNA templates; subjecting the DNA templates and the first complementary copies to DNA glycosylase treatment, wherein the DNA glycosylase specifically excises the modified nucleobase in the DNA templates to convert the positions of the modified nucleobases into abasic sites, resulting in each glycosylase-converted DNA template being hybridized to a non-converted complementary copy; generating a second complementary copy of the glycosylase-converted DNA templates, the generating being directed by a second DNA polymerase, wherein the second DNA polymerase is capable of incorporating a nucleotide opposite the abasic sites in the converted DNA templates, wherein the nucleotide does not Watson-Crick base pair with the modified nucleobase; determining the nucleotide sequence of the first and second complementary copies; and comparing the nucleotide sequence of the second complementary copies to the nucleotide sequence of the first complementary copies for each of the DNA glycosylase-converted DNA templates, thereby determining the positions of the modified nucleobase in the DNA templates prior to DNA glycosylase conversion.

Embodiment 2. The method of embodiment 1, wherein the step of comparing the nucleotide sequence of the second complementary copies to the nucleotide sequence of the first complementary copies for each of the DNA glycosylase-converted DNA templates, identifies a nucleotide substitution in the sequence of the second complementary copies relative to the first complementary copies, wherein the position of the nucleotide substitution identifies the position of the modified base in the DNA templates.

Embodiment 3. The method of embodiment 1 or 2, wherein the modified nucleobase is selected from the group consisting of 5-mC, 5-hmC, 5-fC, and 5-caC.

Embodiment 4. The method of any one of embodiments 1 to 3, wherein the DNA glycosylase is a monofunctional DNA glycosylase.

Embodiment 5. The method of embodiment 4, wherein the monofunctional DNA glycosylase is thymine DNA glycosylase (TDG), or a variant thereof.

Embodiment 6. The method of embodiment 5, wherein the step of subjecting the DNA templates and the first complementary copies to DNA glycosylase treatment further comprises subjecting the DNA templates and the first complementary copies to treatment with a ten eleven translocation (TET) enzyme, or a variant thereof.

Embodiment 7. The method of embodiment 6, wherein the ten eleven translocation (TET) enzyme, or a variant thereof, is ngTET.

Embodiment 8. The method of any one of embodiments 1 to 3, wherein the DNA glycosylase is a bifunctional DNA glycosylase.

Embodiment 9. The method of embodiment 8, wherein the bifunctional DNA glycosylase is member of the DEMETER (DME) family of DNA glycosylases, or a variant thereof.

Embodiment 10. The method of embodiment 9, wherein the member of the DEMETER (DME) family of DNA glycosylases, or a variant thereof, is a variant engineered to inactivate lyase activity.

Embodiment 11. The method of any one of embodiments 1 to 10, wherein the second DNA polymerase is an abasic bypass DNA polymerase.

Embodiment 12. The method of embodiment 11, wherein the abasic bypass DNA polymerase is a DPO4 polymerase, or variant thereof.

Embodiment 13. The method of embodiment 12, wherein the DPO4 polymerase, or variant thereof, is a variant comprising the following mutations: M76W, K78E, E79P, Q82W, Q83G, and S86E (SEQ ID NO:3)

Embodiment 14. The method of any one of embodiments 11 to 13, wherein the abasic bypass DNA polymerase incorporates dATP in the second complementary copies at positions opposite the abasic sites in the glycosylase-converted DNA templates.

Embodiment 15. The method of any one of embodiments 11 to 14, wherein the abasic bypass DNA polymerase further comprises a third DNA polymerase, wherein the third DNA polymerase has exonuclease activity.

Embodiment 16. The method of embodiment 15, wherein the third DNA polymerase is a DPO1 polymerase.

Embodiment 17. The method of any one of embodiments 1 to 16, wherein the first DNA polymerase is a high-fidelity DNA polymerase.

Embodiment 18. The method of any one of embodiments 1 to 17, further comprising the step of treating the glycosylase-converted DNA templates with a stabilizing agent prior to the step of generating the second complementary copies of the glycosylase-converted DNA templates.

Embodiment 19. The method of embodiment 18, wherein the stabilizing agent comprises an aldehyde-reactive compound that forms a stable adduct with the abasic sites.

Embodiment 20. The method of embodiment 19, wherein the stabilizing agent is selected from the group consisting of O-hydroxylamines, acyl hydrazines, tryptamines, beta amino thiols, alkyl hydrazines, hydrazino-iso-pictet-spengler indoles, and methylaminooxy-iso-pictet-spengler indoles.

Embodiment 21. The method of embodiment 19, wherein the stabilizing agent comprises an aminoxyalkyl group capable of forming an oxime adduct with the abasic site.

Embodiment 22. The method of embodiment 21, wherein the stabilizing agent is selected from the group consisting of 1-[2-(amino)ethyl]-uracil, 1-[3-(aminoxy)propyl]-uracil, 1-[4-(aminoxy)butyl]-uracil, 1-[5-(aminoxy)pentyl]-uracil, 1-[2-(aminoxy)ethyl]-2,4-diiodo-5-methyl benzene, 1-[2-(aminoxy)ethyl]-2,4-dibromo-5-methyl benzene, 1-[2-(aminoxy)ethyl]-2,4-dichloro-5-methyl benzene, 1-[2-(aminoxy)ethyl]-2,4-difluoro-5-methyl benzene, and 1-[2-(aminoxy)ethyl]-thymine.

Embodiment 23. The method of embodiment 22, wherein the stabilizing agent is 1-[2-(amino)ethyl]-uracil.

Embodiment 24. The method of embodiment 18, wherein the step of subjecting the DNA templates and the first complementary copies to DNA glycosylase treatment and the step of the treating the glycosylase-converted DNA templates with a stabilizing agent prior to generating the second complementary copies occur in the same step.

Embodiment 25. The method of any one of embodiments 1 to 24, wherein the DNA templates are selected from the group consisting of genomic DNA, mitochondrial DNA, cell-free DNA, circulating tumor DNA, or combinations thereof.

Embodiment 26. The method of any one of embodiments 1 to 25, wherein the DNA templates are immobilized on a solid support.

Embodiment 27. The method of any one of embodiments 1 to 26, wherein the first or second complementary copies are immobilized on a solid support.

Embodiment 28. The method of any one of embodiments 1 to 27, wherein the step of determining the nucleotide sequences of the first and second complementary copies comprises the steps of synthesizing an Xpandomer copy of the first and second complementary copies and passing the Xpandomer copies of the first and second complementary copies through a nanopore.

Embodiment 29. The method of any one of embodiments 1 to 28, wherein the DNA templates comprise a first adapter joined to the 5′ end of the DNA template and a second adapter joined to the 3′ end of the template.

Embodiment 30. The method of embodiment 29, wherein the first or second adapters are Y adapters.

Embodiment 31. The method of embodiment 29, wherein at least one of the first and second adapters comprises a unique molecular identifier barcode (UMI).

Embodiment 32. The method of embodiment 31, wherein the step of comparing the sequences of the first and second complementary copies comprises bioinformatically pairing sequences comprising the same unique molecular identifier barcode (UMI).

Embodiment 33. A chemo-enzymatic nucleobase conversion reaction mixture comprising a DNA glycosylase enzyme, a chemical stabilizing agent, and a suitable buffer.

Embodiment 34. The chemo-enzymatic nucleobase conversion reaction mixture of embodiment 33, further comprising a DNA template strand hybridized to a first complementary copy strand, wherein the DNA template strand comprises a modified nucleobase and the first complementary copy strand comprises native nucleobases.

Embodiment 35. The chemo-enzymatic nucleobase conversion reaction mixture of embodiment 33 or 34, wherein the chemical stabilizing agent comprises an aminoxyalkyl group, wherein the aminoxyalkyl group is capable of reacting with an abasic nucleotide comprising an open-ring aldehyde moiety to form a stable oxime adduct.

Embodiment 36. The chemo-enzymatic nucleobase conversion reaction mixture of embodiment 35, wherein the chemical stabilizing agent is selected from the group consisting of 1-[2-(amino)ethyl]-uracil, 1-[3-(aminoxy)propyl]-uracil, 1-[4-(aminoxy)butyl]-uracil, 1-[5-(aminoxy)pentyl]-uracil, 1-[2-(aminoxy)ethyl]-2,4-diiodo-5-methyl benzene, 1-[2-(aminoxy)ethyl]-2,4-dibromo-5-methyl benzene, 1-[2-(aminoxy)ethyl]-2,4-dichloro-5-methyl benzene, 1-[2-(aminoxy)ethyl]-2,4-difluoro-5-methyl benzene, and 1-[2-(aminoxy)ethyl]-thymine.

Embodiment 37. The chemo-enzymatic nucleobase conversion reaction mixture of embodiment 35, wherein the chemical stabilizing agent is selected from the group consisting of O-hydroxylamines, acyl hydrazines, tryptamines, beta amino thiols, alkyl hydrazines, hydrazino-iso-pictet-spengler indoles, and methylaminooxy-iso-pictet-spengler indoles.

Embodiment 38. The chemo-enzymatic nucleobase conversion reaction mixture of any one of embodiments 33 to 37, wherein the DNA glycosylase is selected from the group consisting of N-methylpurine DNA Glycosylase (MPG), MutY Homolog (MUTYH), Nth-like DNA Glycosylase 1 (NTHL1), Nei-like DNA Glycosylase 1 (NEIL1), Nei-like DNA Glycosylase 2 (NEIL2), Nei-like DNA Glycosylase 3 (NEIL3), 8-oxoguanine DNA glycosylase (OGG1), Uracil DNA Glycosylase 1 (Ung1), Uracil DNA Glycosylase 2 (Ung2), Single-strand selective monofunctional uracil glycosylase (SMUG1), Thymine DNA Glycosylase (TDG), Methyl binding domain 4 (MBD4), Fpg, Ung, Demeter (DME), and ROS1.

Embodiment 39. The chemo-enzymatic nucleobase conversion reaction of mixture of embodiment 38, wherein the reaction mixture comprises more than one DNA glycosylase.

Embodiment 40. The chemo-enzymatic nucleobase conversion reaction mixture of any one of embodiments 33 to 37, wherein the DNA glycosylase is TDG, or a variant thereof.

Embodiment 41. The chemo-enzymatic nucleobase conversion reaction mixture of embodiment 40, further comprising a TET enzyme.

Embodiment 42. A kit for the detection of a modified nucleobase in a DNA sample, comprising the chemo-enzymatic nucleobase conversion reaction mixture of any one of embodiments 33 to 41, an enzyme selected from at least one of a high-fidelity DNA polymerase, an abasic bypass DNA polymerase, and a DNA polymerase with exonuclease activity and a suitable mixture of dNTPs, or analogs thereof.

Embodiment 43. The kit of embodiment 42, further comprising one or more of a buffer for the enzyme.

What is claimed is:

1. A method of identifying a modified nucleobase in a plurality of nucleic acids, the method comprising:
providing a sample comprising a plurality of DNA templates;
generating first complementary copies of the DNA templates, the generating being directed by an oligonucleotide primer using a first DNA polymerase in the presence of native dNTPs, wherein the generating produces a complementary copy of each of the DNA templates such that each complementary copy comprises native dNTPs, and wherein each complementary copy is hybridized to one of the DNA templates;
subjecting the DNA templates and the first complementary copies to DNA glycosylase treatment, wherein the DNA glycosylase specifically excises the modified nucleobase in the DNA templates to convert the positions of the modified nucleobases into abasic sites, resulting in each glycosylase-converted DNA template being hybridized to a non-converted complementary copy;
generating a second complementary copy of the glycosylase-converted DNA templates, the generating being directed by a second DNA polymerase, wherein the second DNA polymerase is capable of incorporating a nucleotide opposite the abasic sites in the converted DNA templates, wherein the nucleotide does not Watson-Crick base pair with the modified nucleobase;
determining the nucleotide sequence of the first and second complementary copies; and
comparing the nucleotide sequence of the second complementary copies to the nucleotide sequence of the first complementary copies for each of the DNA glycosylase-converted DNA templates, thereby determining the positions of the modified nucleobase in the DNA templates prior to DNA glycosylase conversion.

2. The method of claim 1, wherein the step of comparing the nucleotide sequence of the second complementary copies to the nucleotide sequence of the first complementary copies for each of the DNA glycosylase-converted DNA templates, identifies a nucleotide substitution in the sequence of the second complementary copies relative to the first complementary copies, wherein the position of the nucleotide substitution identifies the position of the modified base in the DNA templates.

3. The method of claim 1, wherein the modified nucleobase is selected from the group consisting of 5-mC, 5-hmC, 5-fC, and 5-caC.

4. The method of claim 1, wherein the DNA glycosylase is a monofunctional DNA glycosylase, wherein the monofunctional DNA glycosylase is thymine DNA glycosylase (TDG), or a variant thereof.

5. The method of claim 1, wherein the step of subjecting the DNA templates and the first complementary copies to DNA glycosylase treatment further comprises subjecting the DNA templates and the first complementary copies to treatment with a ten eleven translocation (TET) enzyme, or a variant thereof, and wherein the ten eleven translocation (TET) enzyme, or a variant thereof, is ngTET.

6. The method of claim 1, wherein the DNA glycosylase is a bifunctional DNA glycosylase, wherein the bifunctional DNA glycosylase is member of the DEMETER (DME) family of DNA glycosylases, or a variant thereof.

7. The method of claim 6, wherein the member of the DEMETER (DME) family of DNA glycosylases, or a variant thereof, is a variant engineered to inactivate lyase activity.

8. The method of claim 1, wherein the second DNA polymerase is an abasic bypass DNA polymerase.

9. The method of claim 8, wherein the abasic bypass DNA polymerase is a DPO4 polymerase, or variant thereof, wherein the DPO4 polymerase, or variant thereof, is a variant comprising the following mutations: M76W, K78E, E79P, Q82W, Q83G, and S86E (SEQ ID NO:3), and wherein the abasic bypass DNA polymerase incorporates dATP in the second complementary copies at positions opposite the abasic sites in the glycosylase-converted DNA templates.

10. The method of claim 8, wherein the abasic bypass DNA polymerase further comprises a third DNA polymerase, wherein the third DNA polymerase has exonuclease activity, and wherein the third DNA polymerase is optionally a DPO1 polymerase.

11. The method of claim 1, wherein the first DNA polymerase is a high-fidelity DNA polymerase.

12. The method of claim 1, further comprising the step of treating the glycosylase-converted DNA templates with a stabilizing agent prior to the step of generating the second complementary copies of the glycosylase-converted DNA templates.

13. The method of claim 12, wherein the stabilizing agent comprises an aldehyde-reactive compound that forms a stable adduct with the abasic sites.

14. The method of claim 12, wherein the stabilizing agent is selected from the group consisting of O-hydroxylamines, acyl hydrazines, tryptamines, beta amino thiols, alkyl hydrazines, hydrazino-iso-pictet-spengler indoles, and methylaminooxy-iso-pictet-spengler indoles.

15. The method of claim 12, wherein the stabilizing agent comprises an aminoxyalkyl group capable of forming an oxime adduct with the abasic site.

16. The method of claim 15, wherein the stabilizing agent is selected from the group consisting of 1-[2-(amino)ethyl]-uracil, 1-[3-(aminoxy)propyl]-uracil, 1-[4-(aminoxy)butyl]-uracil, 1-[5-(aminoxy)pentyl]-uracil, 1-[2-(aminoxy)ethyl]-2,4-diiodo-5-methyl benzene, 1-[2-(aminoxy)ethyl]-2,4-dibromo-5-methyl benzene, 1-[2-(aminoxy)ethyl]-2,4-dichloro-5-methyl benzene, 1-[2-(aminoxy)ethyl]-2,4-difluoro-5-methyl benzene, and 1-[2-(aminoxy)ethyl]-thymine.

17. The method of claim 12, wherein the step of subjecting the DNA templates and the first complementary copies to DNA glycosylase treatment and the step of the treating the glycosylase-converted DNA templates with a stabilizing agent prior to generating the second complementary copies occur in the same step.

18. The method of claim 1, wherein the DNA templates are selected from the group consisting of genomic DNA, mitochondrial DNA, cell-free DNA, circulating tumor DNA, or combinations thereof.

19. The method of claim 1, wherein the DNA templates are immobilized on a solid support.

20. The method of claim 1, wherein the first or second complementary copies are immobilized on a solid support.

21. The method of claim 1, wherein the step of determining the nucleotide sequences of the first and second complementary copies comprises the steps of synthesizing an Xpandomer copy of the first and second complementary copies and passing the Xpandomer copies of the first and second complementary copies through a nanopore.

22. The method of claim 1, wherein the DNA templates comprise a first adapter joined to the 5′ end of the DNA template and a second adapter joined to the 3′ end of the template, wherein the first or second adapters are Y adapters, and wherein at least one of the first and second adapters optionally comprises a unique molecular identifier barcode (UMI).


SEQUENCE LISTING (3 sequences)

SEQ ID NO: 1 - protein, 352 aa, Sulfolobus solfataricus
MIVLFVDFDYFYAQVEEVLNPSLKGKPVVVCVFSGRFEDSGAVATANYEARKFGVKAGIPIVEAKKILPNAVYLPMRKEVYQQVSSRIMNLLREYSEKIEIASIDEAYLDISDKVRDYREAYNLGLEIKNKILEKEKITVTVGISKNKVFAKIAADMAKPNGIKVIDDEEVKRLIRELDIADVPGIGNITAEKLKKLGINKLVDTLSIEFDKLKGMIGEAKAKYLISLARDEYNEPIRTRVRKSIGRIVTMKRNSRNLEEIKPYLFRAIEESYYKLDKRIPKAIHVVAVTEDLDIVSRGRTFPHGISKETAYSESVKLLQKILEEDERKIRRIGVRFSKFIEAIGLDKFFDT

SEQ ID NO: 2 - protein, 340 aa, Sulfolobus solfataricus
MIVLFVDFDYFYAQVEEVLNPSLKGKPVVVCVFSGRTELSGAVATANYEARKFGVYSGMPIVRAKKILPNAVYLPWREPVYWGVSERIMNLLREYSEKIEIASIDEAYLDISDKVRDYREAYNLGLEIKNKILEKEKITVTVGISKNKVFAAVAGSKAKPNGIKVIDDEEVKRLIRELNIADVQGIPYFTAQKLKKLGINKLVDTLSIEFDKLKGMIGEAKAKYLISLARDEYNEPIRTRVRKSIGRTVTMKRNSRNLEEIKPYLFRAIEECYYKLDKRIPKAIHVVAWRSRWNSQYRWSWFPHGISKETAYSESVKLLQQILKKDKRKIRRIGVRFSKF

SEQ ID NO: 3 - protein, 340 aa, Sulfolobus solfataricus
MIVLFVDFDYFYAQVEEVLNPSLKGKPVVVCVFSGRFEDSGAVATANYEARKFGVKAGIPIVEAKKILPNAVYLPWREPVYWGVSSRIMNLLREYSEKIEIASIDEAYLDISDKVRDYREAYNLGLEIKNKILEKEKITVTVGISKNKVFAKIAADMAKPNGIKVIDDEEVKRLIRELDIADVPGIGNITAEKLKKLGINKLVDTLSIEFDKLKGMIGEAKAKYLISLARDEYNEPIRTRVRKSIGRIVTMKRNSRNLEEIKPYLFRAIEESYYKLDKRIPKAIHVVAVTEDLDIVSRGRTFPHGISKETAYSESVKLLQKILEEDERKIRRIGVRFSKF